(19) United States
(12) Patent Application Publication
(10) Pub. No.: US 2026/0130751 A1
(43) Pub. Date: May 14, 2026

(54) INSTRUMENTS AND METHODS FOR ANCHORAGE OF SOFT TISSUE

(71) Applicant: Stryker Corporation, Kalamazoo, MI (US)

(72) Inventors: Kyle Craig Pilgeram, San Jose, CA (US); Roger Pisarnwongs, Santa Clarita, CA (US); Nicole Marie Heim, Willmington, NC (US)

(73) Assignee: Stryker Corporation, Kalamazoo, MI (US)

(21) Appl. No.: 19/388,827

(22) Filed: Nov. 13, 2025

Related U.S. Application Data
(60) Provisional application No. 63/720,443, filed on Nov. 14, 2024.

(51) Int. Cl. A61F 2/08

(52) U.S. Cl. CPC A61F 2/0805; A61F 2002/0852; A61F 2002/0882

(57) ABSTRACT

A system for attaching a first tissue to a second tissue that includes an instrument and an implant. The instrument includes a handle, an outer shaft extending from the handle, an inner shaft disposed within the outer shaft and an actuation mechanism configured to control a position of one of the inner shaft and the outer shaft relative to the handle. The implant is releasably attached to a distal end of the instrument and includes a proximal surface that faces a distal end surface of the outer shaft. When a filament is operatively engaged to the implant and the actuation mechanism, a relationship between the proximal surface of the implant and the distal end surface of the outer shaft causes the implant to flip in a predetermined manner upon actuation of the actuation mechanism.

### CROSS-REFERENCE TO RELATED PATENT APPLICATION

This application claims the benefit of and priority to U.S. Provisional Application No. 63/720,443, filed on November 14, 2024, the entire disclosure of which is hereby incorporated by reference herein.

### BACKGROUND

Various surgical tools and techniques have been developed to reattach soft tissue in joints of the body, such as the elbow. In some procedures, an implant may be anchored onto a bone while also securing a soft tissue with the use of a suture. One example of such a procedure is reattachment of a distal biceps to a radius. In such a procedure, an implant and suture may be used to secure the distal biceps to the radius. However, in existing techniques, it is difficult to load suture onto an implant without misplacing the suture or causing the suture to become tangled. Further, it is difficult to maintain an implant in a stable position on a delivery tool when advancing the implant to an implantation location.

Accordingly, a need exists for improved instrumentation and methods of delivery for reattachment and repair of soft tissue in joints of the body.

### BRIEF SUMMARY

The present disclosure provides improved surgical tools, implants, systems and associated methods to secure tissue into or onto a bone. For systems and associated methods, such improvements include flipping of an implant, e.g., button implant, in a predetermined manner when released from an instrument. In one example implementation, a method includes delivery of a button implant with an improved ability to flip the button implant onto a surface of the bone in a predetermined manner.

In a first aspect, the present disclosure relates to a system for attaching soft tissue to a bone. In a first example, a system for attaching soft tissue to a bone includes an instrument and a button implant. The instrument includes a handle, a movable component on the handle, and a shaft extending from the handle. The movable component is configured to control a portion of the shaft. The button implant is releasably attached to a distal end of the shaft. Further, manipulation of the movable component on the handle causes the button implant to be released from the instrument such that an end of the button implant remote from the instrument flips to a first or a second side of a central longitudinal axis of the shaft in a predetermined manner.

In a second example, the portion of the shaft of the first example is a first portion of the shaft, and the first portion of the shaft may be slidably disposed within a second portion of the shaft. In a third example, the system of the first example may include a retention filament removably engaged to the button implant and the movable component, the retention filament configured to pull the button implant releasably attached to the distal end of the shaft when the movable component is moved away from the button implant. In a fourth example, the retention filament of the third example may pass through a first eyelet of the button implant and a second eyelet of the button implant, and first and second end portions of the retention filament are clamped by the movable component when the button implant is attached to the distal end of the shaft.

In a fifth example, the system of the first example may be arranged such that a central longitudinal axis along an elongate dimension of the button implant is angled relative to the central longitudinal axis of the shaft. In a sixth example, the system of the fifth example may be arranged such that the elongate dimension of the button implant extends from a first end to a second end, the second end configured to attach to the shaft and having a surface at an oblique angle relative to a surface along the elongate dimension. In a seventh example, the system of the fifth example may be arranged such that the elongate dimension of the button implant extends from a first end to a second end, the second end including an opening therein sized to receive the portion of the shaft. In an eighth example, the system of the first example may be arranged such that the button implant has an elongate dimension extending from a first end surface to a second end surface, the second end surface including an opening therein sized to receive the shaft of the instrument, where a central longitudinal axis of the opening is non-parallel to a central longitudinal axis along the elongate dimension of the button implant. In a ninth example, the system of the first example may be arranged such that the handle includes an opening sized to receive a soft tissue and measure the soft tissue, the opening being transverse to a length direction of the handle.

In a tenth example, the system of any one of the first through ninth examples may be configured such that the handle may define a plurality of openings, each opening of the plurality of openings passing through opposing outer surfaces of the handle such that each opening of the plurality of openings is oriented transverse to a length direction of the handle. The plurality of openings may be sized to receive a soft tissue and measure the soft tissue. In an eleventh example, the system of the tenth example may be configured such that at least two openings of the plurality of openings in the handle have a different maximum cross-sectional dimension. In a twelfth example, the system of any one of the first through eleventh examples may include a sleeve configured to releasably attach onto the shaft of the handle such that the sleeve remains in position relative to the shaft when attached to the shaft. In a thirteenth example, the system of the twelfth example may be configured such that the sleeve includes a slot oriented along a length direction of the sleeve.

The system of the first aspect may also have other configurations. In a first example of one such alternative configuration, a system includes an instrument, an implant and a loading tab. The instrument includes a shaft, and the implant is releasably attached to a distal end of the shaft. The implant includes a first opening and a second opening spaced apart from the first opening. The loading tab is movably received on the shaft and includes a first wire and a second wire received thereon. When the loading tab is positioned over the implant, a first working filament is attachable to the first wire and a second working filament is attachable to the second wire so that when the loading tab is pulled away from the implant, the first working filament passes through the first opening and the second opening, and the second working filament passes through the first opening and the second opening in a different manner than the first working filament. In variations of the first example, the loading tab may be pulled proximally along the shaft away from the implant, diagonally away from the implant, or in a direction orthogonal to an elongate dimension of the instrument away from the implant.

In a second example of the alternative configuration, the system of the first example may be arranged such that the second opening is closer to the distal end of the shaft than the first opening and when the loading tab is pulled proximally along the shaft, the second working filament initially enters the second opening and the first working filament initially enters the first opening. In a third example, the system of the first example may be arranged such that the loading tab includes a plurality of channels therein, a first channel from among the plurality of channels receiving a first wire therein and a second channel from among the plurality of channels receiving a second wire therein, respective first ends of the first and second wires being attached to the loading tab. In a fourth example, the system of the third example may be arranged such that the plurality of channels of the loading tab are shaped so that when the loading tab is pulled proximally along the shaft, the first working filament passes through the first opening then the second opening and the second working filament passes through the second opening then the first opening. In a fifth example, the system of the first example may be arranged such that the loading tab includes a tab body and an extension such that when the tab body is positioned in a distal portion of the shaft, at least a portion of the extension is closer to the implant than the shaft. In a sixth example, the system of the first example may be arranged such that a second end of each of the first and second wires is an enclosed loop and/or a length of the first wire is different from the second wire. In a seventh example, the system of the first example may be arranged such that the first wire and the second wire are made of a metal material (e.g., nitinol, stainless steel, titanium, etc.) or a suture material (e.g., multifilament suture, braided multifilament suture, monofilament suture, etc.). In an eighth example, the system of the first example may be arranged such that when the loading tab is positioned over the implant, the first working filament is attachable to the first wire and the second working filament is attachable to the second wire. In such arrangement, when the loading tab is pulled proximally along the shaft away from the implant, the first working filament passes through the first opening and the second opening, and the second working filament passes through the first opening and the second opening in a different manner than the first working filament.

In yet another configuration of the first aspect, a system is configured for attaching a first tissue to a second tissue. In a first example, the system includes an instrument and an implant. The instrument includes a handle, an outer shaft, an inner shaft and an actuation mechanism. The outer shaft extends from the handle and includes a distal end surface. The inner shaft extends from the handle and is disposed within the outer shaft. The actuation mechanism is configured to control a position of one of the inner shaft and the outer shaft relative to the handle. The implant is releasably attached to a distal end of the instrument and includes a proximal surface that faces the distal end surface of the outer shaft. The system is configured such that when a filament is operatively engaged to the implant and the actuation mechanism, a relationship between the proximal surface of the implant and the distal end surface of the outer shaft causes the implant to flip in a predetermined manner upon actuation of the actuation mechanism.

In a second example, the system of the first example may be configured such that at least one of the proximal surface of the implant and the distal end surface of the outer shaft includes a chamfered surface. In a third example, the system of the first example may be configured such that the proximal surface of the implant includes two planar surfaces separated by an edge extending across the proximal surface, the implant being configured to flip about an axis along the edge. In a fourth example, the system of the first example may be configured such that at least one of the proximal surface of the implant and the distal end surface of the outer shaft includes a protrusion thereon. In a fifth example, the system of the first example may be configured such that the implant is releasably attached to the distal end of the instrument has a central axis along an elongate dimension of the implant that is at an angle relative to a central longitudinal axis of the outer shaft. In a sixth example, the system of the first example may be configured such that the implant is releasably attached to the distal end of the instrument has a central axis along an elongate dimension of the implant that is at an angle relative to a central longitudinal axis of the outer shaft and at least part of the proximal surface of the implant is angulated relative to the distal end surface of the outer shaft. In a seventh example, the system of the sixth example may be configured such that the implant defines an opening and is configured to receive a distal tip of the inner shaft, a central longitudinal axis of the opening being transverse to the central axis of the implant. In a variation of this example, the opening extends from the proximal surface of the implant.

In an eighth example, the system of the first example may be configured such that the implant releasably attached to the distal end of the instrument has a volumetric center that is offset from a central longitudinal axis of the outer shaft and at least part of the proximal surface of the implant is angulated relative to the distal end surface of the outer shaft. In a ninth example, the system of the first example may be configured such that the implant releasably attached to the distal end of the instrument has a volumetric center that is offset from a central longitudinal axis of the outer shaft such that an opening defined by the implant and extending from the proximal surface of the implant is closer to one of an upper surface of the implant and a lower surface of the implant, the proximal surface extending between the upper surface and the lower surface, and the opening being configured to receive a tip of the inner shaft. In a tenth example, the system of the first example may include the filament. The filament may extend on a single side of the implant and the outer shaft between the implant and the actuation mechanism. In an eleventh example, the system of the first example may be configured such that the actuation mechanism includes a handle tab configured such that translation of the handle tab along a length direction of the handle causes a simultaneous translation of the inner shaft or the outer shaft.

In a second aspect, the present disclosure relates to a kit for attaching soft tissue to a bone. In a first example, a kit for attaching soft tissue to a bone includes an instrument, a retention filament and a button implant. The instrument includes a handle and a shaft extending therefrom. The retention filament is configured to be movably attached to the instrument. And, the button implant has an elongate dimension extending from a first end to a second end, a surface at the first end including an opening therein. The button implant is configured to be attached to the shaft. When the instrument is used to deliver the button implant into a patient, the retention filament is attached to the button implant and a tip of the shaft is received in the opening of the button implant.

In a second example, the kit of the first example may be arranged such that the shaft is an inner shaft and the instrument includes an outer shaft such that the inner shaft is movable relative to the outer shaft. In this example, the inner shaft may be configured such that when the button implant is disposed on the inner shaft and the retention filament passes through at least one eyelet of the button implant and is attached to the instrument, retraction of the inner shaft relative to the outer shaft causes the button implant to detach from the inner shaft and flip relative to the instrument. In a variation of this example, the button implant is held by the outer shaft and the inner shaft is configured to advance axially out of the outer shaft to cause the button implant to detach from the outer shaft and flip relative to the instrument.

In a third example, the kit of the first example may be arranged such that the shaft is an outer shaft and the instrument includes an inner shaft such that the outer shaft is movable relative to the inner shaft. In one variation, with a retention filament passed through at least one eyelet of a button implant attached to the instrument, the outer shaft may be configured to be advanceable relative to the inner shaft such that upon sufficient advancement, the button implant, previously attached to the inner shaft, is caused to be flipped in a predetermined manner. In another variation, the outer shaft may be configured such that when the button implant is disposed on the outer shaft and the retention filament passes through at least one eyelet of the button implant and is attached to the instrument, retraction of the outer shaft relative to the inner shaft causes the button implant to detach from the outer shaft and flip relative to the instrument.

In a fourth example, the kit of the first example may be arranged such that the button implant includes a first eyelet and a second eyelet, the retention filament being configured for passage through each of the first eyelet and the second eyelet. In a fifth example, the kit of the first example may be arranged such that a central axis of the opening of the button implant is at a non-zero angle relative to a central axis of the elongate dimension of the button implant. In a sixth example, the kit of the first example may be arranged such that the surface at the first end of the button implant is at an oblique angle relative to a surface along the elongate dimension of the button implant. In a seventh example, the kit of the first example may include a loading tab configured for attachment to the shaft of the instrument. The loading tab may include a plurality of channels therein, at least a subset of the plurality of channels receiving a wire. And, when the button implant and the loading tab are attached to the shaft, and a working filament is attached to the wire, moving of the loading tab in a direction away from the button implant causes the working filament to pass through one or more eyelets of the button implant in a predetermined manner. In one variation of the seventh example, moving of the loading tab away from the button implant may be sliding of the loading tab in a proximal direction. In an eighth example, the kit of the first example may include a loading tab configured for attachment to the shaft of the instrument. The loading tab may include a plurality of channels therein, at least a subset of the plurality of channels receiving one of a first wire and a second wire. When the first and second wires of the at least the subset of the plurality of channels are passed through the button implant and a working filament is attached to the first and second wires, moving of the loading tab away from the button implant causes the working filament to pass through one or more eyelets of the button implant in a predetermined manner.

In a ninth example, the kit of the first example may include a drill pin configured for the formation of a hole through a bone of the patient, the hole being formed in preparation for receipt of the instrument therethrough. In a tenth example, the kit of the first example may include a working filament. In an eleventh example, the kit of the ninth example may include a needle configured for use with the working filament.

In a third aspect, the present disclosure relates to a method of attaching a soft tissue to a bone. In a first example, a method includes: passing first and second end portions of a working filament engaged to a soft tissue through one or more eyelets of a button implant releasably attached to a distal end of a shaft of an instrument; advancing the instrument into a hole in a bone; manipulating an actuation mechanism on the instrument to cause a first elongate surface of the button implant to flip onto a bone surface of the bone as the button implant detaches from the shaft, wherein the first elongate surface is configured to flip with respect to the bone surface in a predetermined manner and a second elongate surface opposite the first elongate surface is configured to flip so that the second elongate surface faces away from the bone surface; and securing the first and second end portions of the working filament to stabilize the button implant. Securement may be via a knot against the button implant or passage of the working filament back into the soft tissue, among other approaches.

In a second example, the shaft is an inner shaft, and the method of the first example may include pulling the actuation mechanism proximally relative to a handle of the instrument as part of the manipulating of the actuation mechanism to cause the inner shaft to retract relative to an outer shaft of the instrument housing the inner shaft until the distal end of the inner shaft detaches from the button implant, and then pulling further such that a retention filament passing through one or more eyelets of the button implant and attached to the instrument causes the button implant to flip. In a third example, the method of the second example may include, before the distal end of the shaft detaches from the button implant, drawing a trailing surface of the button implant toward an end of the outer shaft such that when the trailing surface contacts the outer shaft, an angulation of the trailing surface relative to a central axis of the shaft induces the button implant to flip so that the first elongate surface faces the bone surface, the angulation of the trailing surface being at an oblique angle relative to the first elongate surface. In a fourth example, the manipulation of the actuation mechanism in the second example is an initial manipulation and the method of the second example may include, subsequent to the initial manipulation, subsequently manipulating the actuation mechanism to cause the retention filament to be released from its attachment to the instrument. In a fifth example, the method of the fourth example may be performed such that the initial manipulation may include sliding the actuation mechanism away from the distal end of the shaft and the subsequent manipulation comprises rotating the actuation mechanism.

In a sixth example, the method of the first example may be performed such that, prior to the passing aspect, the button implant is releasably attached to the shaft such that a central axis along an elongate dimension of the button implant is at an acute angle relative to a central longitudinal axis of the shaft. In a seventh example, the method of the first example may include: prior to the passing aspect, attaching the respective first and second end portions of the working filament to respective first and second wires attached to a loading tab, the loading tab being attached to the shaft; and sliding the loading tab in a proximal direction along the shaft until the first and second end portions of the working filament each pass through one or more eyelets of the button implant. In an eighth example, the method of the seventh example may include attaching the respective first and second end portions of the working filament such that the first and second end portions are attached to respective first and second closed loops at ends of the first and second wires. In a ninth example, the method of the seventh example may include use of first and second wires such that the first wire and the second wire have different lengths, and when sliding the loading tab in the proximal direction, an end of the first wire passes through the button implant at a different time than an end of the second wire. In a tenth example, the method of the first example may include measuring a diameter of the soft tissue by passing a portion of the soft tissue through an opening in a handle of the instrument. Such measurement may be used to determine a reamer size for forming a hole in the bone to receive the soft tissue. In an eleventh example, the method of the first example may be performed on an elbow where the bone is a radial bone and the soft tissue is a biceps muscle. In a twelfth example, the method of the first example may include advancing the instrument into the hole in the bone until the button implant is located outside of the bone on an opposite side of the bone from which the instrument entered the bone. In a thirteenth example, the method of the first example may include manipulating the actuation mechanism such that the first elongate surface of the button implant flips onto the bone surface where the bone surface is within the hole in the bone.

### BRIEF DESCRIPTION OF THE DRAWINGS

The features, aspects, and advantages of the present disclosure will become better understood with reference to the following description, appended claims, and accompanying drawings in which:

FIG. 1A is a perspective view of a system according to one example of the present disclosure;

FIG. 1B is a perspective view of a system according to another example of the present disclosure;

FIG. 2 is a side cross-sectional view of the system of FIG. 1A;

FIG. 3 is a top cross-sectional view of a handle of a system according to one example of the present disclosure;

FIG. 4 is a perspective cross-sectional view of the system of FIG. 1A;

FIG. 5 is a close-up partial perspective cross-sectional view of a system according to one example of the present disclosure;

FIG. 6 is a close-up partial perspective view of the shafts and some components of the actuation mechanism of an instrument of a system according to one example of the present disclosure;

FIG. 7A is a close-up view of a distal end portion of the system of FIG. 1A;

FIG. 7B is a perspective view of a loading tab of the system of FIG. 1A;

FIG. 8A is a bottom view of a distal end portion of the system of FIG. 1B;

FIG. 8B is a perspective view of a loading tab of the system of FIG. 1B;

FIG. 9 is a perspective view of a loading tab of the system of FIG. 1A;

FIG. 10 is a perspective view of another loading tab accordingly to one example of the present disclosure;

FIG. 11 is a perspective view of another loading tab according to one example of the present disclosure;

FIG. 12A is a perspective view of a button implant according to one example of the present disclosure;

FIG. 12B is a side view of the button implant of FIG. 12A;

FIG. 13A is a perspective view of a button implant according to one example of the present disclosure;

FIG. 13B is a sectional view from the side the button implant of FIG. 13A;

FIG. 14 is an illustration of an exemplary aspect in a method of preparing a system to attach soft tissue to a bone according to one example of the present disclosure;

FIG. 15 is an illustration of an exemplary aspect in a method of preparing a system to attach soft tissue to a bone according to one example of the present disclosure;

FIG. 16 is an illustration of an exemplary aspect in a method of preparing a system to attach soft tissue to a bone according to one example of the present disclosure;

FIG. 17 is an illustration of an exemplary aspect in a method of preparing a system to attach soft tissue to a bone according to one example of the present disclosure;

FIG. 18 is an illustration of an exemplary aspect in a method of preparing a system to attach soft tissue to a bone according to one example of the present disclosure;

FIG. 19 is an illustration of an exemplary aspect in a method of preparing a system to attach soft tissue to a bone according to one example of the present disclosure;

FIG. 20 is an illustration of an exemplary aspect in a method of preparing a system to attach soft tissue to a bone according to one example of the present disclosure;

FIG. 21 is an illustration of an exemplary aspect in a method of preparing a system to attach soft tissue to a bone according to one example of the present disclosure;

FIG. 22 is an illustration of an exemplary aspect in a method of preparing a system to attach soft tissue to a bone according to one example of the present disclosure;

FIG. 23 is an illustration of an exemplary aspect in a method of preparing a system to attach soft tissue to a bone according to one example of the present disclosure;

FIG. 24 is an illustration of an exemplary aspect in a method of preparing a system to attach soft tissue to a bone according to one example of the present disclosure;

FIG. 25 is an illustration of an exemplary aspect in a method of preparing a system to attach soft tissue to a bone according to one example of the present disclosure;

FIG. 26 is an illustration of an exemplary aspect in a method of preparing a system to attach soft tissue to a bone according to one example of the present disclosure;

FIG. 27 is an illustration of an exemplary aspect in a method of preparing a system to attach soft tissue to a bone according to one example of the present disclosure;

FIG. 28 is an illustration of an exemplary aspect in a method of preparing a system to attach soft tissue to a bone according to one example of the present disclosure;

FIG. 29A is an illustration of an exemplary aspect in a method of preparing a system to attach soft tissue to a bone according to one example of the present disclosure;

FIG. 29B is an illustration of an exemplary aspect in a method of preparing a system to attach soft tissue to a bone according to one example of the present disclosure;

FIG. 29C is an illustration of an exemplary aspect in a method of preparing a system to attach soft tissue to a bone according to one example of the present disclosure;

FIG. 29D is an illustration of an exemplary aspect in a method of preparing a system to attach soft tissue to a bone according to one example of the present disclosure;

FIG. 30 is an illustration of an exemplary aspect in a method of attaching soft tissue to a bone according to one example of the present disclosure;

FIG. 31 is an illustration of an exemplary aspect in a method of attaching soft tissue to a bone according to one example of the present disclosure;

FIG. 32 is an illustration of an exemplary aspect in a method of attaching soft tissue to a bone according to one example of the present disclosure;

FIG. 33 is an illustration of an exemplary aspect in a method of attaching soft tissue to a bone according to one example of the present disclosure;

FIG. 34 is an illustration of an exemplary aspect in a method of attaching soft tissue to a bone according to one example of the present disclosure;

FIG. 34A is an illustration of an exemplary aspect in a method of attaching soft tissue to a bone according to one example of the present disclosure;

FIG. 35 is an illustration of an exemplary aspect in a method of attaching soft tissue to a bone according to one example of the present disclosure;

FIG. 36 is an illustration of an exemplary aspect in a method of attaching soft tissue to a bone according to one example of the present disclosure;

FIG. 37 is an illustration of an exemplary aspect in a method of attaching soft tissue to a bone according to one example of the present disclosure;

FIG. 38 is an illustration of an exemplary aspect in a method of attaching soft tissue to a bone according to one example of the present disclosure;

FIG. 39A is an illustration of an exemplary aspect in a method of attaching soft tissue to a bone according to one example of the present disclosure;

FIG. 39B is an illustration of an exemplary aspect in a method of attaching soft tissue to a bone according to one example of the present disclosure;

FIG. 39C is an illustration of an exemplary aspect in a method of attaching soft tissue to a bone according to one example of the present disclosure;

FIG. 40 is an illustration of an exemplary aspect in a method of attaching soft tissue to a bone according to one example of the present disclosure;

FIG. 41 is an illustration of an exemplary aspect in a method of attaching soft tissue to a bone according to one example of the present disclosure;

FIG. 42 is an illustration of an exemplary aspect in a method of attaching soft tissue to a bone according to one example of the present disclosure;

FIG. 43 is a side cross-sectional view of an instrument handle according to one example of the present disclosure;

FIG. 44 is an illustration of an exemplary aspect in a method of operating the instrument handle of FIG. 43, according to one example of the present disclosure;

FIG. 45 is an illustration of an exemplary aspect in a method of operating the instrument handle of FIG. 43, according to one example of the present disclosure;

FIG. 46 is an illustration of an exemplary aspect in a method of operating the instrument handle of FIG. 43, according to one example of the present disclosure;

FIG. 47 is a side view of a suture adapted for use with a loading tab according to one example of the present disclosure;

FIG. 48 is an illustration of an exemplary aspect in a method of using an instrument with a sleeve according to one example of the present disclosure; and

FIG. 49 is an illustration of an exemplary aspect in a method of using an instrument with a sleeve according to one example of the present disclosure.

### DETAILED DESCRIPTION

As used herein, "proximal" or "proximally" means closer to or towards an operator, e.g., a surgeon, while "distal" or "distally" means further from or away from the operator. As used herein, the terms “substantially,” “generally,” “approximately,” and “about” are intended to mean that slight deviations from absolute are included within the scope of the term so modified.

The instrumentation and other surgical aids contemplated by the present disclosure may be used to deliver and place an implant onto or into a bone of a patient, the implant holding soft tissue of the patient via a filament, e.g., suture. Soft tissue that may be repaired and otherwise reattached using the instrumentation of the present disclosure may be, for example, a distal biceps tendon in the elbow, a proximal biceps tendon in the shoulder, ligaments that stabilize the clavicle, a pectoralis tendon in the chest, a ligament in the knee, tissue in the hand or wrist, ankle syndesmosis and other tissue proximate to the ankle.

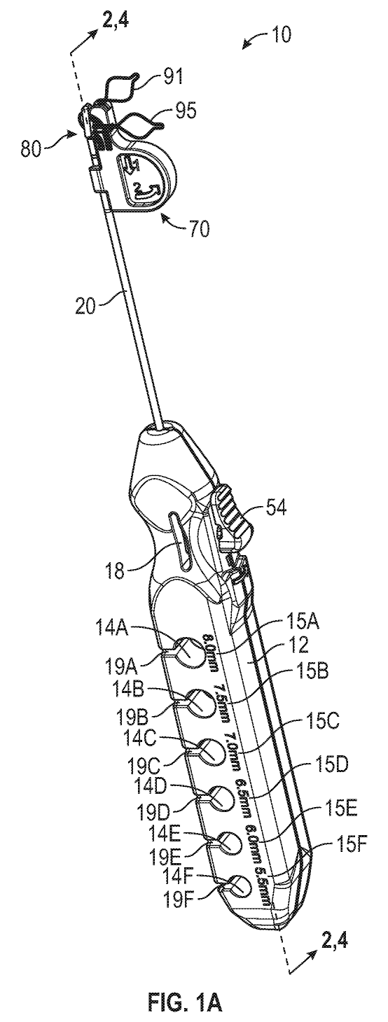

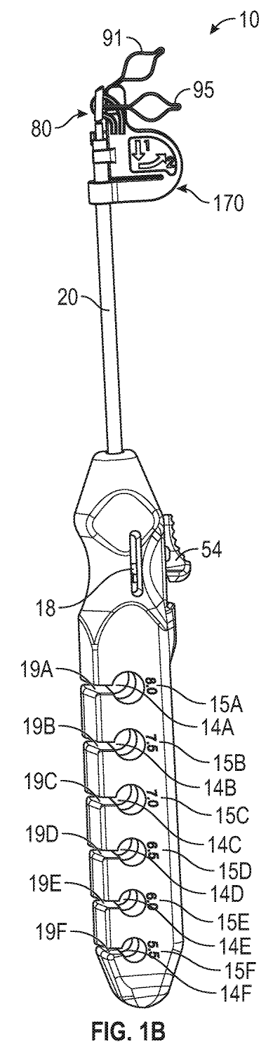

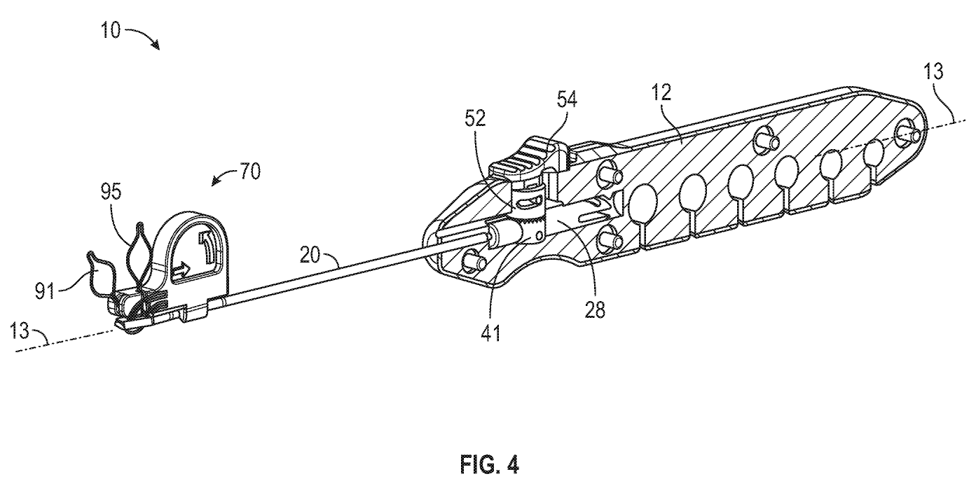

In one aspect, the present disclosure relates to an instrument for loading and delivering a working filament. Instrument 10, shown in FIGS. 1A-2 and 4-6, is one example of the instrument. Instrument 10 includes a handle 12 and an outer shaft 20 that extends from handle 12. In one example, and as depicted, a central longitudinal axis 13 (indicated in FIG. 4) is centered along handle 12 and outer shaft 20. And, as depicted, outer shaft 20 is fixed relative to handle 12. In some examples, and in the depicted example, outer shaft 20 is cannulated.

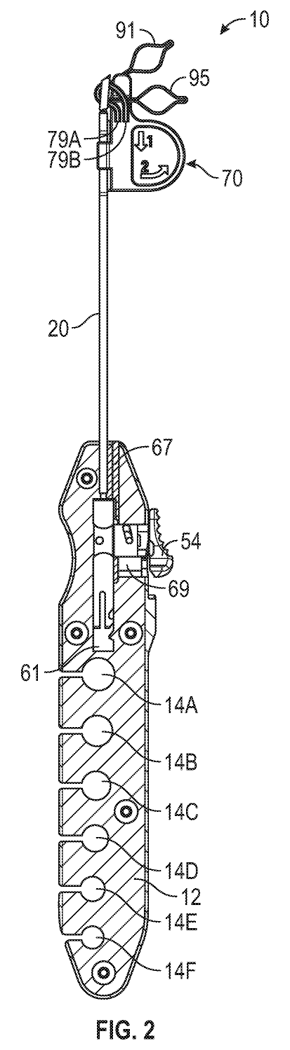

Handle 12 is shaped to define a set of measurement openings 14A-F, each extending entirely through handle 12 between opposing outer surfaces of handle 12 and being separate from the others, as shown in FIGS. 1A-2. Each measurement opening of the set of measurement openings 14A-F has a different size. In some examples, and as depicted, the set of measurement openings 14A-F are circular in cross-sectional shape such that each measurement opening is cylindrical over a distance between opposing sides of handle 12 with the exception of a gap at an interface with a respective slit 19A-F, described in greater detail below. Further, the set of measurement openings 14A-F includes openings ranging in size from 5.5 mm in diameter to 8.0 mm in diameter in 0.5 mm increments. Handle 12 may optionally include size indicators for the measurement openings, such as markings 15A-15F shown in FIGS. 1A-1B. Handle 12 is also shaped to define a set of slits 19A-F that are in communication with respective measurement openings 14A-F on one side of the set of separate measurement openings 14A-F. Each measurement opening of the set of measurement openings 14A-F may receive a soft tissue therein for purposes of evaluating a size of such soft tissue. The availability of different measurement opening sizes defined on handle 12 allows a user to easily identify a soft tissue size based on matching the soft tissue with the best-fitting measurement opening. Slits 19A-F provide additional access to the set of measurement openings 14A-F such that a filament may be passed from the side of the handle through a slit and into a measurement opening in communication with the slit. In this manner, when a soft tissue is positioned through a measurement opening, a filament may be passed through a slit and then into the soft tissue.

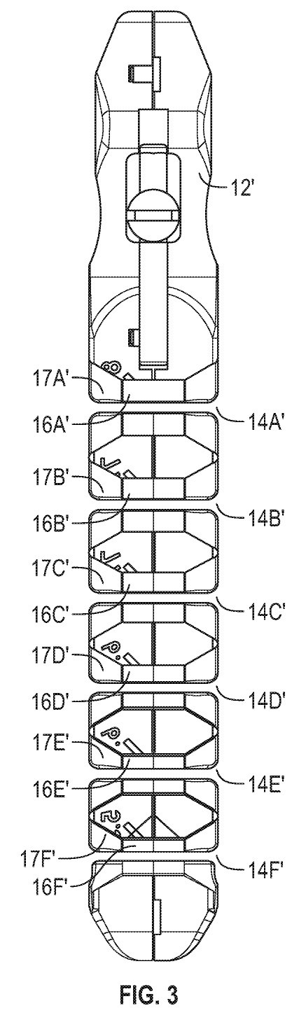

In an alternative example, an instrument such as instrument 10 shown in FIGS. 1A-2 may include a handle 12´ as shown in a top-down section view in FIG. 3. Reference numerals in the 10´-series of numerals for handle 12´ refer to like elements in the 10-series of numerals for handle 12 unless otherwise indicated. Handle 12´ defines a set of measurement openings 14A´-14F´. Each individual measurement opening of the set of measurement openings 14A´-14F´ includes a central portion 16A´-16F´ that separates tapered portions 17A´-17F´ on opposing sides of central portion 16A´-16F´. Tapered portions 17A´-17F´ are analogous to a funnel in that a size of a measurement opening from among the set of measurement opening 14A´-14F´ is largest on its opposing peripheries and tapers to the central portion 16A´-16F´. Central portion 16A´-16F´ may be cylindrical in shape with a gap at an interface with the respective slit 19A´-19F´. In variations, surfaces of the tapered portions may be flat. In other variations, surfaces of the tapered portions may be curved in two directions to define a spherical-type surface. Further, the inclusion of a tapered portion on opposing sides of the measurement openings of the set of measurement openings 14A´-14F´ is advantageous in that a user may access a measurement opening without the need for excessive manipulation of handle 12´. For instance, a user may actively attempt to compress the soft tissue into a smaller cylinder, i.e., measurement opening, than could be achieved without the leading tapered portion. It should be understood that, throughout the disclosure, any reference to handle 12 as part of an instrument 10, or any system, kit or method using handle 12, may be interchangeable with handle 12´.

In other examples, a cross-sectional shape of the openings of set of measurement openings 14A-14F, 14A´-14F may be ovular or have another non-circular cross-sectional shape. In some of these examples, two or more openings on a handle may have different cross-sectional shapes, e.g., different maximum cross-sectional dimensions. In some examples, sizes of the openings of the set of measurement openings 14A-14F, 14A´-14F may vary from that shown in handle 12 and handle 12´. In still further examples, the handle may have any number of measurement openings on handle 12 or handle 12´, such as one, two, three or more openings.

Returning to instrument 10 shown in FIGS. 1A-2 and 4-6, we now turn to the manipulatable features of instrument 10. A distal portion of handle 12 includes a shaft receiving cavity 61 extending from a distal end of handle 12 that is aligned along central longitudinal axis 13 of the handle, as shown in FIG. 4. A portion of outer shaft 20 and a portion of inner shaft 24 are disposed within shaft receiving cavity 61. Shaft receiving cavity 61 includes a narrow distal part and a wider proximal part, i.e., has a narrower diameter in the distal part than in the proximal part. As depicted, outer shaft 20 is immovably fixed within the distal part of shaft receiving cavity 61. At least part of a distal portion 25 of an inner shaft 24 is disposed within a lumen of outer shaft 20 and is slidable relative to outer shaft 20. As described in greater detail elsewhere in the present disclosure, in a distal most advancement of inner shaft 24 relative to outer shaft 20, inner shaft 24 projects from a distal end surface 22 of outer shaft 20, and in a proximal most withdrawal of inner shaft 24 relative to outer shaft 20, a tip of inner shaft 24 is enclosed within outer shaft 20.

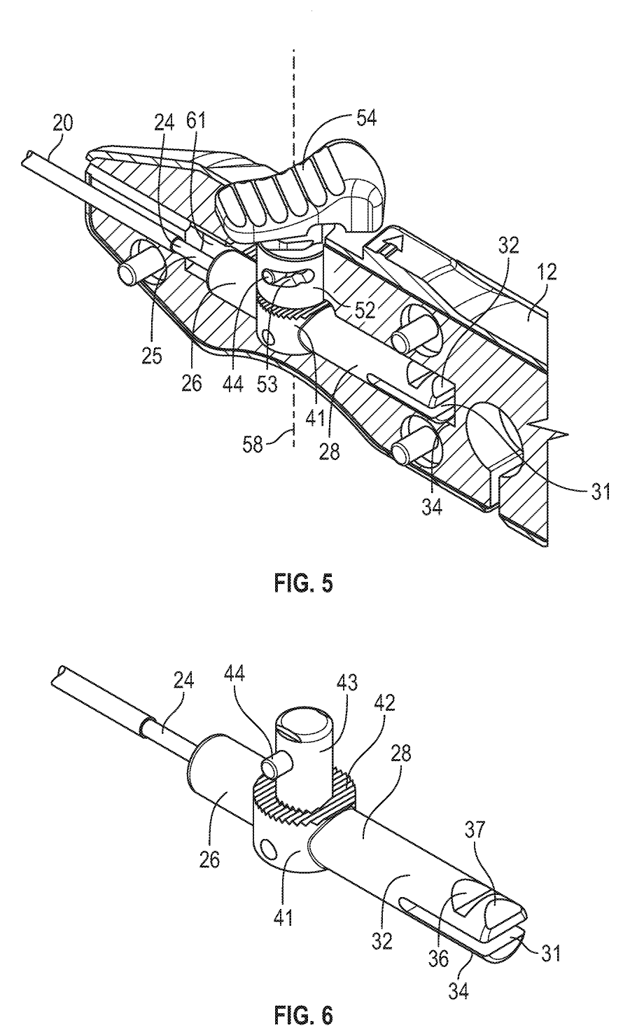

Inner shaft 24 includes, from a first end to an opposite second end, distal portion 25, translation-limiting portion 26, lower locking block 41 and terminal portion 28. In some examples, inner shaft 24 may be a solid rod. In other examples, inner shaft 24 may be cannulated. Each aforementioned component of inner shaft 24 is fixed with respect to the others such that when inner shaft 24 translates relative to outer shaft 20, each component translates together. As depicted, distal portion 25, translation-limiting portion 26 and terminal portion 28 are cylindrically shaped with centers generally aligned with central longitudinal axis 13 of handle 12. Lower locking block 41 includes an engagement surface 42 facing a handle tab 54, as shown in FIGS. 5-6. Engagement surface 42 may have a friction enhancing surface, such as teeth (as shown), individual protrusions, or a roughened surface, among other surface types. Lower locking block 41 has a different shape compared to adjacent shaft portions to accommodate engagement surface 42. With continued reference to the components of inner shaft 24, and as shown in FIG. 6, inner block 43 extends from engagement surface 42 toward handle tab 54, and pin 44 extends radially from a side of inner block 43. Inner block 43 is smaller in cross-section than lower locking block 41. Lower locking block 41, inner block 43 and pin 44 are fixed together. In some examples, and as depicted, lower locking block 41, inner block 43 and pin 44 have cylindrical shapes. Terminal portion 28 includes a slit 31 separating a first end prong 32 from a second end prong 34. First and second end prongs 32, 34 have a capacity for flexure and may bend into a space of slit 31. First end prong 32 includes a groove 36 and a radial projection 37 for interaction with internal surfaces of shaft receiving cavity 61 at predetermined locations. Specifically, an inner wall of handle 12 defining shaft receiving cavity 61 includes positioning projection 64A that are complementary to groove 36, as shown in FIG. 36. In this manner, when inner shaft 24 is translated relative to shaft receiving cavity 61, a position of inner shaft 24 may be set in a predetermined location based on engagement of groove 36 with a positioning projection 64A.

With continued reference to instrument 10, a handle tab 54 and an upper locking block 52 fixed thereto are in operative communication with inner block 43. Inner block 43 is received in a lumen of upper locking block 52, and pin 44 projects through slot 53 of upper locking block 52. When assembled, upper locking block 52 may be partially or entirely internal to handle 12, while handle tab 54 may be accessible from locations external to handle 12 for ease of access by a user. Due to the manner in which inner shaft 24 is disposed within shaft receiving cavity 61, pin 44 maintains a relatively constant rotational position inside handle 12, i.e., pin 44 does not rotate relative to a locking axis 58, while handle tab 54 and upper locking block 52 are rotatable together relative to a locking axis 58. With this arrangement, rotation of handle tab 54 causes upper locking block 52 to move toward or away from lower locking block 41 while slot 53 slides over pin 44. A length and angle of slot 53 may dictate an extent to which upper locking block 52 may be separated from lower locking block 41. As depicted, when an elongate dimension of handle tab 54 is aligned with an elongate dimension of handle 12, upper locking block 52 is engaged with lower locking block 41. And, when handle tab 54 is rotated so that handle tab 54 is transverse to handle 12 as shown in FIG. 5, upper locking block 52 is disengaged from and spaced apart from lower locking block 41. As depicted, when upper locking block 52 is engaged with lower locking block 41, translation of handle tab 54 relative to handle 12 causes simultaneous translation of inner shaft 24. Collectively, handle tab 54 and upper locking block 52 define an upper portion of an actuation mechanism and inner block 43 and lower locking block 41 define a lower portion of the actuation mechanism, the upper and lower portions combined functioning as one example of an actuation mechanism. It should be appreciated that these principles may be applied in different ways with variations on the components shown in the depicted examples. For example, in some variations, handle tab 54 may be designed so that orientations of handle tab 54 that represent engagement and disengagement with lower locking block 41 are the opposite of that shown in the depicted example. In other variations of the instrument, an actuation mechanism may include components other than a handle tab 54, such as a dial to control translation of the slidable shaft of the instrument.

Components of instrument 10 may be made of biocompatible materials such as metals and polymers. In some examples, inner shaft 24 and outer shaft 20 are made of stainless steel, titanium or nitinol. In some examples, handle 12, and optionally one or more subcomponents thereof, may be made of a polymeric material. Polymeric materials used may include, for example, acrylonitrile butadiene styrene, polycarbonate, or combinations thereof.

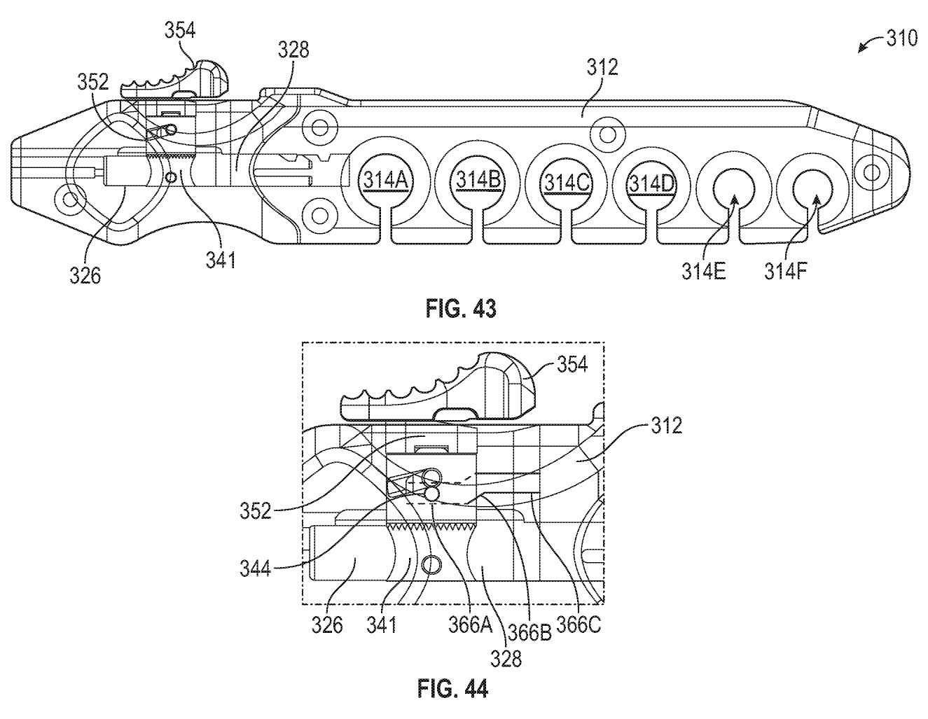

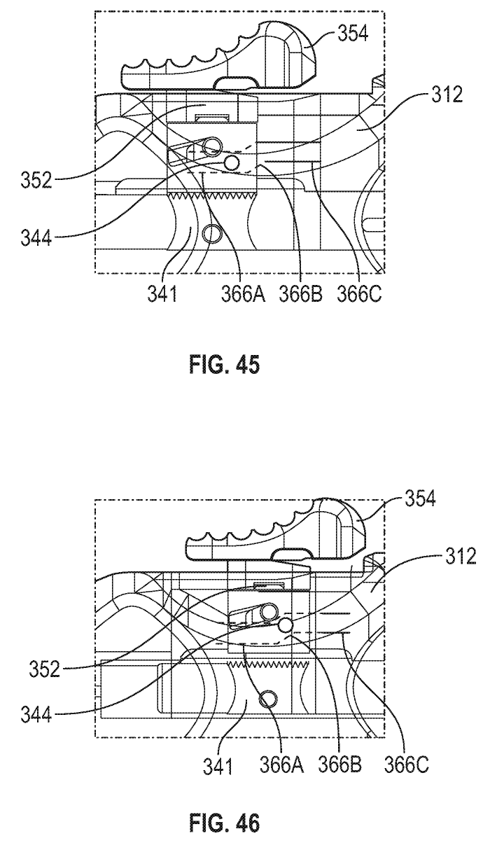

In another example, an instrument 310 is as shown in FIGS. 43-46. Reference numerals in the 300-series of numerals for instrument 310 refer to like elements in the 10-series of numerals for instrument 10 unless otherwise indicated. Instrument 310 includes a handle 312 and an inner shaft partially disposed in handle 312. Handle 312 includes a set of measurement openings 314A-F along a portion of a length of handle 312. The inner shaft includes a distal portion, translation-limiting portion 326, lower locking block 341 and terminal portion 328 (distal portion not shown). Operatively engageable with inner shaft is handle tab 354 and upper locking block 352. In instrument 310, a pin 344 is fixed to upper locking block 352. Further, an inner cavity within handle 312 includes a groove encompassing a lower groove 366A, a transition groove 366B and an upper groove 366C. As the inner shaft is initially retracted proximally into handle 312, upper locking block 352 remains engaged with lower locking block 341 as pin slides along lower groove 366A, as shown in FIGS. 44-45. Then, once handle tab 354 is retracted sufficiently, pin 344 slides along transition groove 366B and into upper groove 366C, and upper locking block 352 is disengaged from lower locking block 341, as shown in FIG. 46. In this manner, only translation of handle tab 354 is required to control both a translational position of the inner shaft relative to handle 312, e.g., for tensioning a retention filament held between lower locking block 341 and upper locking block 352, as described in greater detail elsewhere in the present disclosure, and for engaging and disengaging such retention filament. Put another way, no rotation of handle tab 354 is required to control whether upper locking block 352 is engaged with lower locking block 341.

In yet another example, an instrument may be similar to instrument 10, but with an arrangement where inner shaft 24 is fixed relative to handle 12 and outer shaft 20 slides relative to handle 12 (not shown). In one variation of such a configuration, outer shaft 20 is fixed to lower locking block 41 and handle tab 54 such that translation of the handle tab 54 simultaneously translates outer shaft 20. In other respects, this alternative example may include any number of the features of instrument 10. Operationally, a system with a translatable outer shaft 20 may be configured so that in a delivery position, i.e., holding an implant on instrument 10, outer shaft 20 is retracted, and conversion to a released position, i.e., flipping and releasing the implant from the instrument, is caused by axially extending outer shaft 20 relative to inner shaft 24 to push outer shaft 20 against the implant. In still further examples, an instrument may include an actuatable push button (not shown) within and at a proximal end of shaft receiving cavity 61. In these examples, handle tab 54 is slid proximally in a locked configuration, i.e., upper locking block 52 and lower locking block 41 are engaged, and remains in a locked configuration until contacting the push button. It should be appreciated that the instrument 10 of this arrangement is configured so that button implant 80 is caused to flip prior to handle tab 54 being retracted sufficiently to contact the push button. In terms of the release action, upon further retraction of handle tab 54 after causing the button implant 80 to flip, contact of terminal portion 28 of inner shaft 24, i.e., a proximal end of inner shaft 24 with the push button causes upper locking block 52 to be released from lower locking block 41, thereby releasing a retention filament 102 pinched therebetween. In still further examples, instrument 10 may include a handle tab 54 arranged to translate in a direction that is different from an elongate dimension of handle 12, i.e., not proximally-distally.

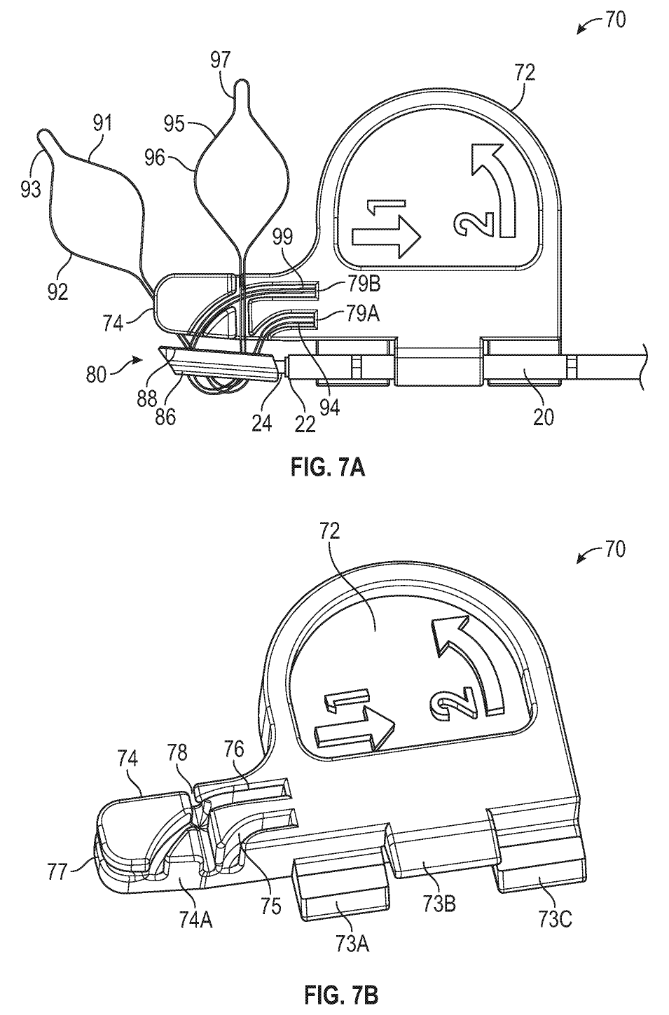

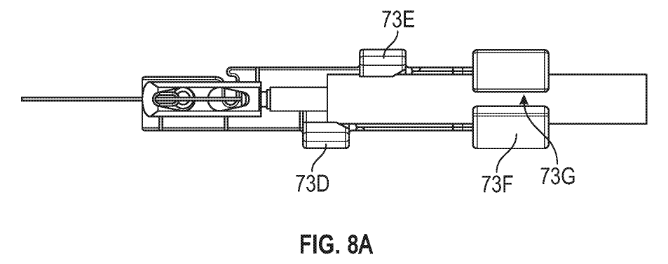

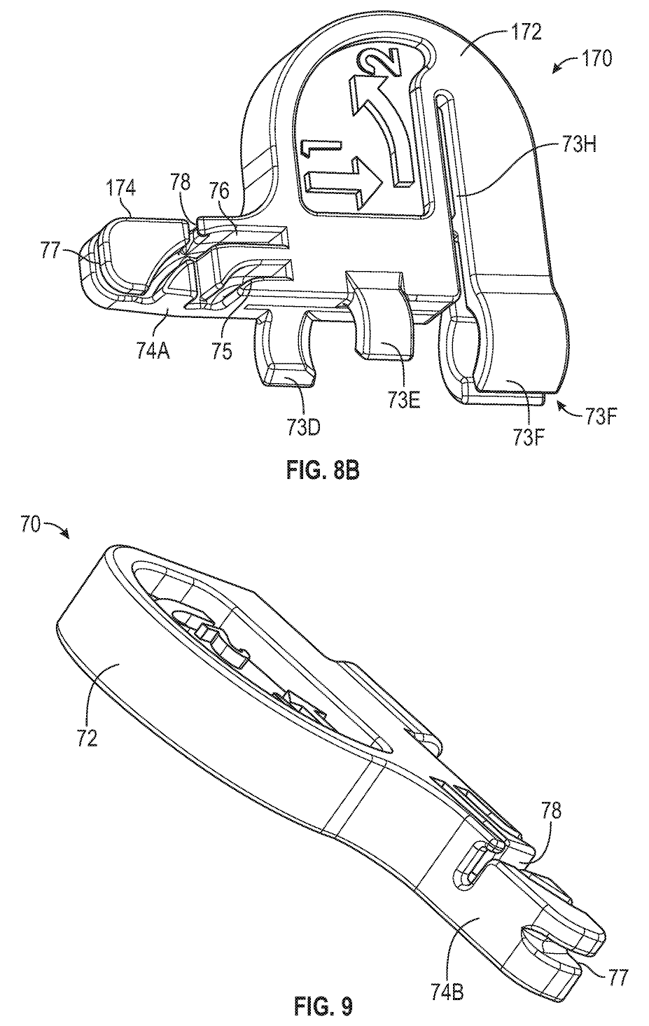

In another aspect, the present disclosure relates to a loading tab configured for use in conjunction with instrument 10. In one example, a loading tab 70, 170, shown in FIGS. 7A-8B is attachable to outer shaft 20 as shown in FIGS. 1A-1B, 2, 4 and 7. As shown in FIGS. 7A-8B, the loading tab 70, 170 includes a body 72, 172 and an extension 74, 174, 174 that extends from body 72, 172. With reference to loading tab 70, body 72 may include a wide and/or flat region for ease of holding and manipulation. As depicted, body 72 has a generally flat shape with a rounded side to minimize a risk of damaging gloves worn by a user of the instrumentation. Extending from one side of body 72 are first, second and third gripping projections 73A-C. Each gripping projection 73A-C defines a groove or channel sized to receive outer shaft 20 of instrument 10. In some examples, and as depicted in at least FIG. 7B, first and third gripping projections 73A, 73C may face a first direction and second gripping projection 73B may face a second direction opposite the first. Such arrangement may enhance removable engagement of loading tab 70 to outer shaft 20.

In another example, a loading tab 170 shown in FIGS. 8A-8B is also attachable to outer shaft 20, for example as shown in at least FIG. 1B. As shown in FIGS. 8A-8B, the loading tab 170 includes a body 172 and an extension 174 that extends from the body 172. The body 172 is shown to include a plurality of gripping projections. As shown in FIGS. 8A-8B, the body 172 includes a first projection 73D, a second projection 73E, and a third projection 73F. In an example embodiment, the projections are arranged to define one or more grooves or channels, which are sized to receive the outer shaft 20 of the instrument 10. For example, the projections 73D and 73E may be arranged to define a first groove or channel sized to receive the outer shaft 20 of the instrument 10, and/or the third projection 73F may be arranged to define a second groove or channel sized to receive the outer shaft 20 of the instrument 10.

In some examples, and as shown in at least FIG. 8A-8B, the first projection 73D extends from a bottom of the body 172 and faces a first direction, and the second projection 73E extends from the bottom of the body 172 and faces a second direction opposite the first direction. Such an arrangement may define a channel or groove between the projections 73D-73E, for example to enhance the engagement between the loading tab 170 to the outer shaft 20. As also shown in FIGS. 8A-8B, the first projection 73D and the second projection 73E are offset, and positioned toward the extension 74 (e.g., in a distal direction, etc.). The third projection 73F is shown extending from the body 172, offset from the second projection 73E, and/or positioned away from the extension 174 (e.g., in a proximal direction, etc.).

As shown in at least FIG. 8B, in some examples the third projection 73F is a cylindrically-shaped projection having a channel 73G extending therethrough. In some examples, the channel 73G formed in the third projection 73F is aligned with the channel formed between the projections 73D-73E, for example to enhance the engagement between the loading tab 70 and the outer shaft 20. As shown in FIG. 8A, the third projection 73F also includes a groove The groove is shown to extend through a bottom portion of the third projection 73F (e.g., to the channel 73G, etc.), for example to allow opposing arms or extensions of the third projection 73F to bias. In some examples, the groove 73G allows the opposing arms of the third projection 73F to bias to allow the channel of the third projection 73F to receive the outer shaft 20. In some examples, the third projection 73F and/or the channel formed therein is sized such that the third projection 73F maintains an engagement with (e.g., is coupled with, attached to, etc.) the outer shaft 20, for example loading tab 170 may maintain its rotational and longitudinal orientation when and/or as the inner shaft 24 moves, as described herein. In some examples, the first projection 73D and/or the second projection 73E are similarly configured to maintain engagement with the outer shaft 20.

As discussed herein, in some examples the loading tab 170 includes additional and/or different components. For example, and as shown in FIGS. 8B, in some examples the body 72 defines or includes a channel or groove, shown as channel 73H. As shown in FIG. 8B, the channel 73H extends from a bottom portion of the body 72, and/or into the body 72. The channel 73H may be positioned adjacent one or more of the projections, for example the third projection 73F, to for example allow portions or sections of the loading tab 70 to bias. For example, during removal and/or manipulation of the loading tab 170 relative to the outer shaft 20, the channel 73H may allow a user to dislodge the outer shaft 20 from the first and second projections 73D, 73E, which may then help the user twist or otherwise move the loading tab 170 relative to the shaft to then dislodge the outer shaft 20 from the third projection 73F.

As shown in FIGS. 7A-8B, extension 74, 174 extends from one side of body 72, 172 and is shaped to define a plurality of channels 75, 76, 77, 78 therein. First and second channels 75, 76 are adjacent each other and curve from an interior location adjacent to body 72, 172 to an inward surface 74A of extension 74. Third channel 77 is within an end surface of extension 74, 174 remote from body 72, 172 and extends between inward surface 74A and outward surface 74B. Fourth channel 78 extends from inward surface 74A to outward surface 74B and crosses second channel 76 along its length, as shown in FIGS. 7B and 9. Each channel is a groove in a surface of extension 74, 174 and does not pass entirely through a thickness of extension 74, 174. It should be appreciated that the depicted example of an arrangement of channels is not limiting, and that other arrangements are also contemplated. Loading tab 70, 170 may be made of biocompatible materials. For example, loading tab 70, 170 may be made of a polymeric material. Polymeric materials used may include, among others, acrylonitrile butadiene styrene, polycarbonate, or a combination thereof. In other examples, loading tab 70, 170 may be made of nylon.

With continued reference to loading tabs 70, 170, plurality of channels 75, 76, 77, 78 are configured to receive, and loading tab 70, 170 may include, first and second wires 91, 95 that function as shuttles for a working filament as described in the methods of the present disclosure. First wire 91 extends from base end 94 to first narrowed end 93. More specifically, and as shown in FIG. 7A, first wire 91 extends from a base end 94 fixed to body 72, 172, through first channel 75, then outside of loading tab 70, 170, and finally along third channel 77, before exiting loading tab 70, 170, and terminating at first narrowed end 93. Proximate first narrowed end 93 and external to extension 74, 174, first wire 91 includes a first loop 92, which bulges relative to first narrowed end 93. As depicted, two ends of first wire 91 pass through respective channels within extension 74, 174, and into body 72 of loading tab 70, 170 at base end 94. Similarly, second wire 95 extends from a base end 99 fixed to body 72, 172, through second channel 76, then outside of loading tab 70, 170, and finally through fourth channel 78, before exiting loading tab 70, 170 and terminating at second narrowed end 97. Proximate second narrowed end 97 and external to extension 74, 174, second wire 95 includes a second loop 96, which bulges relative to second narrowed end 97. As depicted, two ends of second wire 95 pass through respective channels within extension 74, 174 and into body 72 at base end 99. Base ends 94, 99 of respective first and second wires 91, 95 may be extend through respective wire openings 79A, 79B within body 72, as shown in FIGS. 2, 7A, and 8A, for fixation to loading tab 70, 170. First and second wires 91, 95 may be made of a metal or metallic material, such as nitinol, stainless steel braided wire, etc. or a suture or suture material, such as a multifilament suture, a braided multifilament suture, a monofilament suture, or other similar materials.

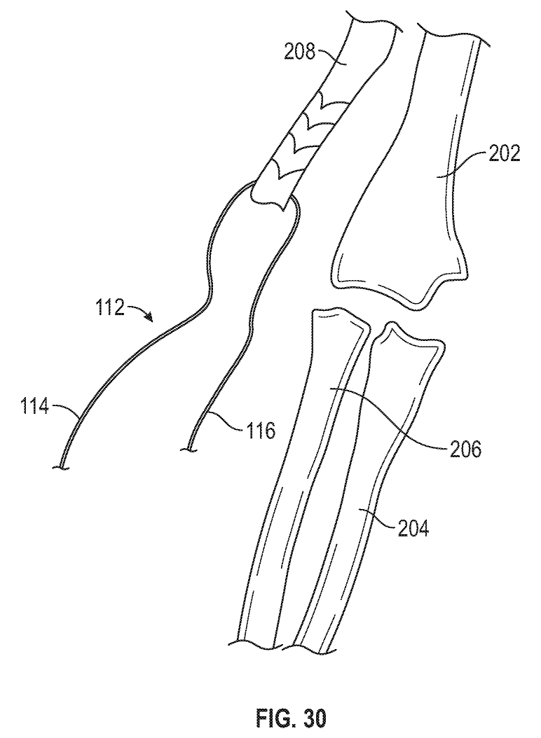

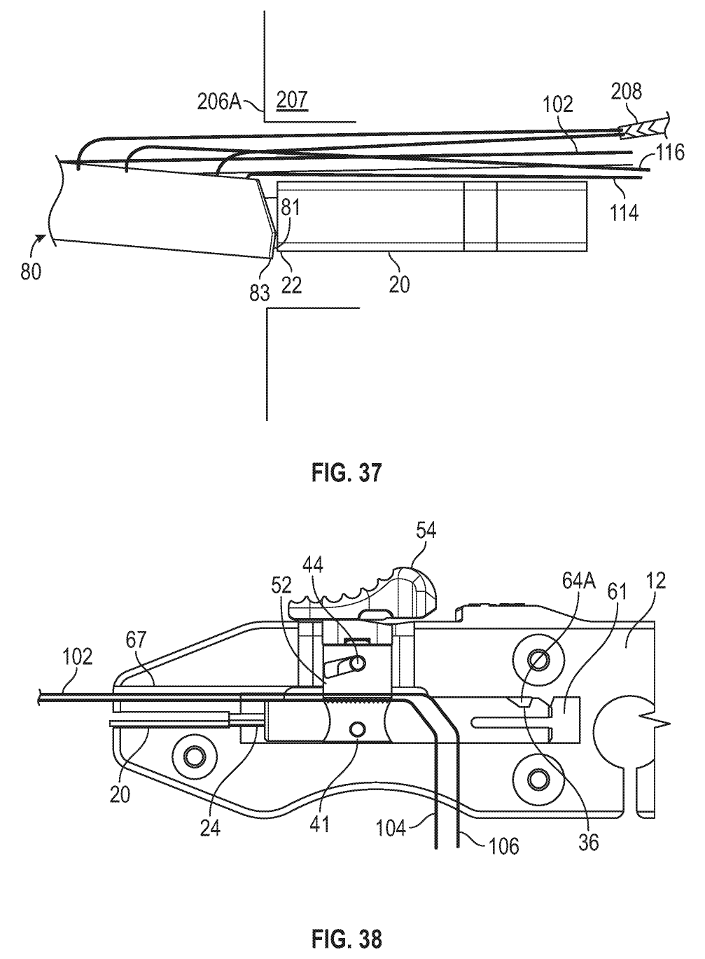

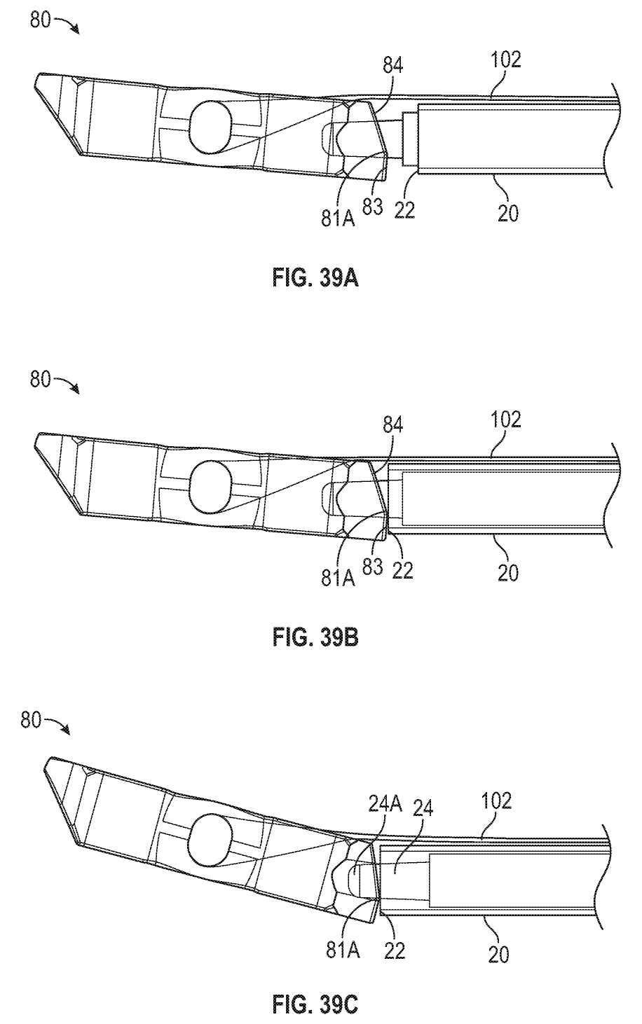

Loading tab(s) 70, 170 provide(s) many advantages when used with instrument 10. For example, extension 74, 174 covers an area over a button implant 80 engaged at a distal end of instrument 10, e.g., as shown in FIG. 7A, to prevent filament, such as working filament 112 (see FIGS. 30-42), from becoming misplaced during deployment. Further, plurality of channels 75, 76, 77, 78 organize first and second wires 91, 95 and aid in the maintenance of loop separation, i.e., keeping first loop 92 and second loop 96 spaced apart. And, usage of loading tab 70, 170 improves a line of sight to wires 91, 95. Plurality of channels 75, 76, 77, 78 also facilitate predictable criss-cross passage of the end portions of working filament 112 through first and second eyelets 85A, 85B of button implant 80 because of a predetermined arrangement of wires 91, 95 through eyelets 85A, 85B, as shown in FIG. 7A, and described in greater detail elsewhere in the present disclosure. Moreover, when loading tab 70, 170 is positioned on instrument 10 and wires 91, 95 receive respective end portions of working filament 112, loading tab 70, 170 mitigates a risk that such end portions of working filament 112 would unintentionally go through the wrong eyelet or through existing loops proximate button implant 80, e.g., a loop of retention filament 102, when end portions of working filament 112 are passed through button implant 80. Yet another advantage of loading tab 70, 170 with wires is that first and second loops 92, 96 on respective first and second wires 91, 95 simplify a process of reloading new filament. When filament is engaged onto a first loop 92 or a second loop 96, such filament is well fixed and the risk of the filament disengaging from first loop 92 or second loop 96 is mitigated. Overall, loading tab 70, 170 functions to automate a process of loading a filament, such as working filament 112, through an implant such as button implant 80.

In some variations of loading tab 70, 170 that interacts with the first and second wires 91, 95, one or both of first and second wires 91, 95 may have a different shapes and/or configuration. As an example, the first wire 91 may be arranged such that the first wire 91 has one or more portions that are coupled to one another. For example, the first wire 91 may extend through wire opening 79A from the base end 94 to the first narrowed end 93 (e.g., through the channels 75, 77, etc.) and back to the base end 94. The first wire 91 may be arranged such that overlapping portions of the first wire 91 are coupled to one another, at least within wire opening 79A towards a first loop 92 (e.g., within the channel 75, the channel 77, and/or outside the loading tab, etc.), to form a coupled (or similar) configuration. It should be understood that it is contemplated that one or more portions of the first wire 91 may be coupled in any suitable arrangement and/or configuration.

Similarly, the second wire 95 may be arranged such that the second wire 95 has one or more portions that are coupled to one another. For example, the second wire 95 may be arranged such that overlapping portions of the second wire 95 (e.g., within the channel 76, the channel 78, and/or outside the loading tab, etc.) are coupled to one another to form a coupled (or similar) configuration, for example at least within wire opening 79B towards a second loop 96. It should be understood that it is contemplated that one or more portions of the second wire 95 may be coupled in any suitable arrangement and/or configuration.

Advantageously, such a coupled (or similar) configuration of one or both of the first or second wires 91, 95 may prevent filament, such as a working filament 112 (see FIGS. 30-42), from becoming positioned between overlapping portions of the first and/or second wires during deployment (e.g., within the channels, etc.). In this sense, such a wire configuration of the wires 91, 95 may advantageously ensure that a working filament does not get positioned, routed, and/or caught between overlapping portions of wires 91, 95 upon deployment, which could result in a tangle of the working filament.

Further, it is contemplated that in other embodiments, the first wire 91 and/or the second wire 95 may be otherwise arranged, configured, and/or shaped. For example, the first wire 91 and/or the second wire 95 may be a single wire or filament having a loop. In this sense, the first wire 91 and/or the second wire 95 may have a loop at one end (e.g., forming a shape similar to the loops 92, 96, etc.), and a tail extending therefrom. The loop may be formed via any suitable technique, for example as a knot, via a splice (e.g., a Brummel splice, etc.) and/or any other suitable arranged. Advantageously, by implementing the first wire 91 and/or the second wire 95 as a single wire or filament having a loop at one end and a tail extending therefrom, the wires 91, 95 advantageously ensure that a working filament does not get positioned, routed, and/or caught around portions wires 91, 95 upon deployment, which could result in a tangle of the working filament, as described herein.

As another example, rather than having a closed loop (i.e., first loop 92 and second loop 96), the wire may have a hook end with a curled tip. Other end shapes are also envisioned, and in particular shapes that facilitate engagement by an end portion of a filament, such as working filament 112. In other variations, a length of one wire may be longer than the other. For example, first wire 91 may be shorter than second wire 95 or vice versa. Relative lengths of first and second wires 91, 95 may be established so that when respective first and second loops 92, 96 and filament end portions pass through eyelets, they do so at different times while loading tab 70 is translated proximally along outer shaft 20.

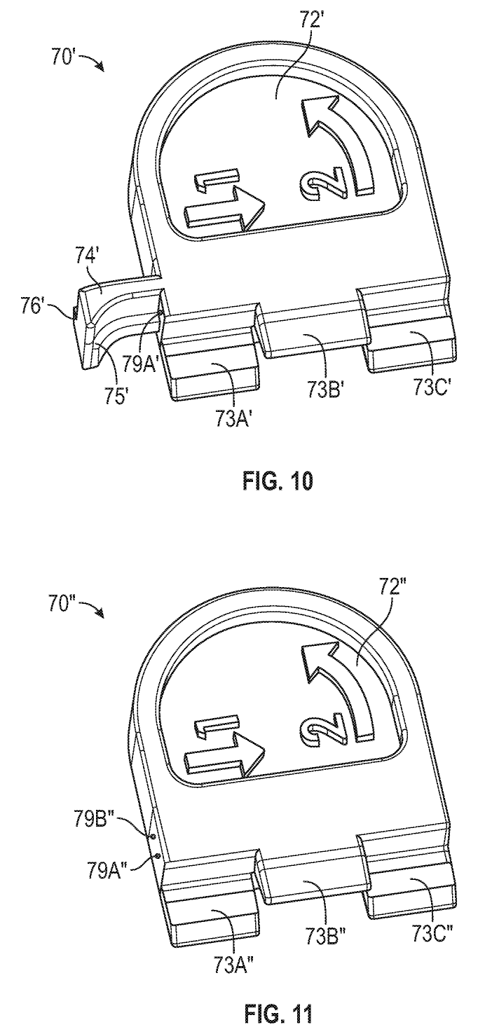

In another example, a loading tab 70` is shown in FIG. 10. Reference numerals in the 70`-series of numerals for loading tab 70` refer to like elements in the 70-series of numerals for loading tab 70 unless otherwise indicated. Loading tab 70` includes body 72` and extension 74` that extends from one side of body 72`. In loading tab 70`, extension 74` does not include any channels, but does include a first track 75` and a second track 76` on opposite sides of extension 74`, such tracks being defined by a stepped surface. In this manner, when wires are attached to loading tab 70`, the respective wires may be positioned on respective first and second tracks 75`, 76`. A shape of first track 75` may be similar to first channel 75 in loading tab 70, and a shape of second track 76` may be similar to a portion of second channel 76 in loading tab 70. Wire openings (one wire opening 79A` shown) extend into body at ends of respective first and second tracks 75`, 76` for receipt of wires (not shown).

In another example, a loading tab 70`` is shown in FIG. 11. Reference numerals in the 70``-series of numerals for loading tab 70`` refer to like elements in the 70-series of numerals for loading tab 70 unless otherwise indicated. Loading tab 70`` includes body 72`’ and does not include any extension. Loading tab 70`` includes wire openings 79A``, 79B`` on a side of body 72`` for receipt of wires (not shown). Loading tabs 70`, 70`` provide greater versatility during use due to the less restrictive nature of the structure. However, a user may exercise greater care to avoid entanglement of wires 91, 95 when using such loading tabs.

In variations of any one of loading tabs 70, 70`, 70``, the body 72, 72`, 72`` of the loading tab may include any number of gripping projections or another similar structure for snap-on and slidable engagement with an outer shaft 20 of instrument 10.

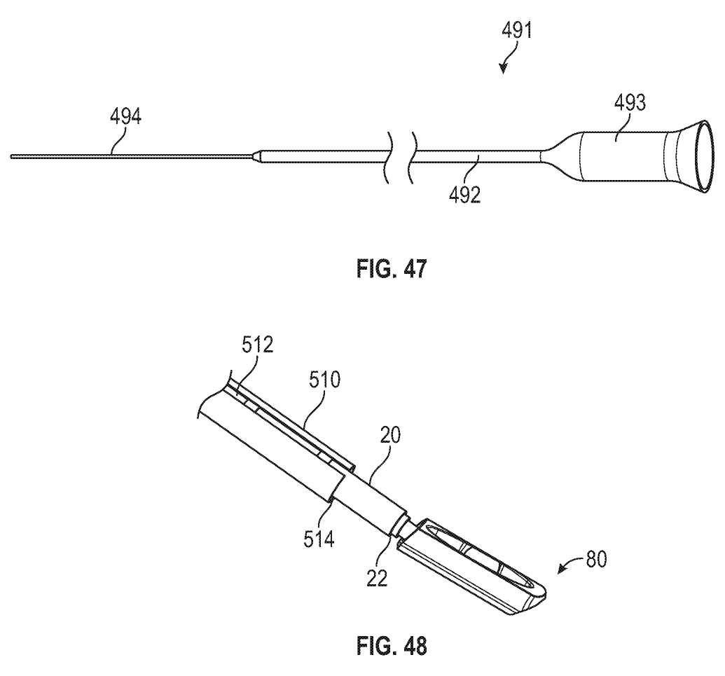

In a variation of any one of the above examples, a loading tab may be configured to receive two shuttles where shuttle 491 as shown in FIG. 47 is representative. In such variations, shuttles 491 are fixed to the loading tab in place of wires 91, 95. Each shuttle 491 includes a stiff end portion 494, a central portion 492 extending from stiff end portion 494 and a hollow end portion 493 extending from central portion 492. Similar to wire 91, 95, stiff end portion 494 may be set in place when positioned through channels 75, 76, 77, 78 of extension 74 such that a segment of stiff end portion 494 of shuttle 491 that is within one or more channels 75, 76, 77, 78 is self-supporting within such channels without holding hollow end portion 493. The hollow end portion 493 may be formed by heat setting a suture material into an expanded shape larger than the central portion 492 to allow for easy loading which is subsequently followed by a collapsing of either central portion 492 and/or hollow end portion 493 to grip onto a loaded repair tail like a finger trap. A repair tail may be an end portion of a filament such as first end portion 114 or second end portion 116 of working filament 112 described elsewhere in the present disclosure, for example. In variations, stiff end portion 494 may be removed from a remainder of shuttle 491 after manufacture but before use. In still further variations, shuttle 491 may be formed without a stiff end portion 494.

In another aspect, the function of the loading tab 70 may be provided without any loading tab. Specifically, the wires 91, 95 may be provided without a loading tab while still being used as shuttles for a working filament. Such wires 91, 95 may be secured to each other at one end using a variety of means, such as knotting the wires together or a simple biocompatible structure that receives each wire 91, 95.

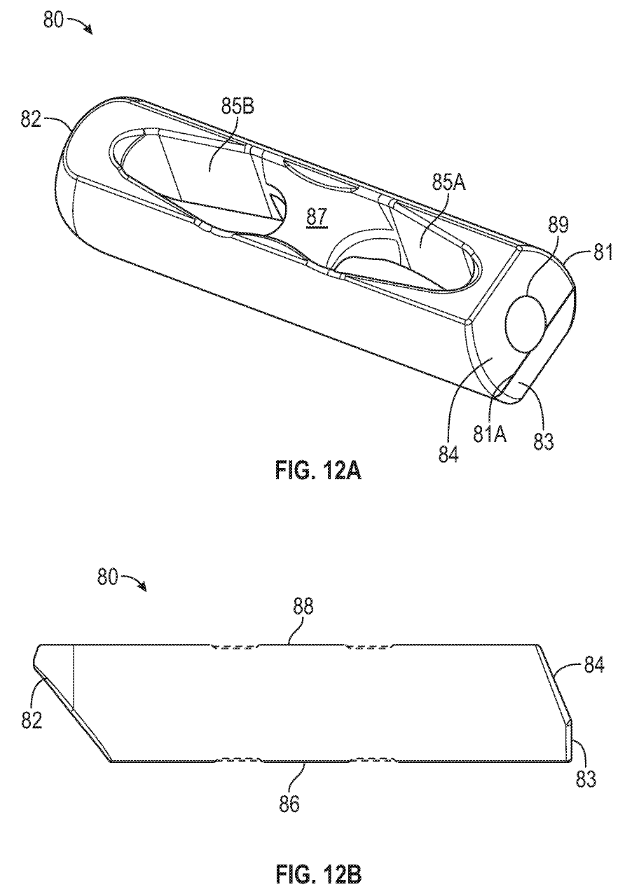

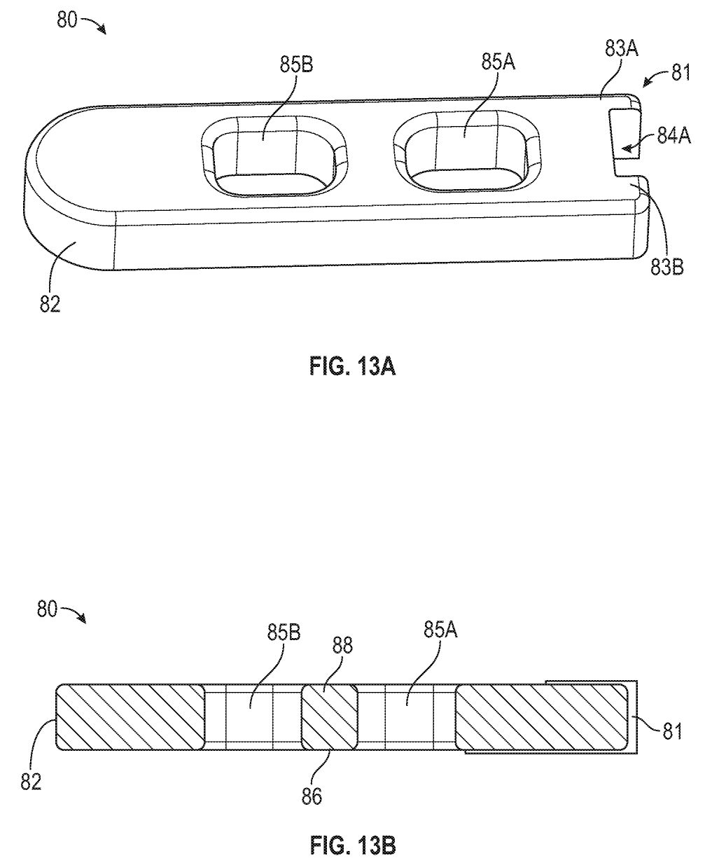

In another aspect, the present disclosure relates to an implant for use as part of a repair of a soft tissue in a patient. In one example, an implant is a button implant 80. As shown in at least FIGS. 12A-13B, the button implant 80 has a length extending from a first end 81 to a second end 82, and is configured to be anchored in or on bone and to secure soft tissue to button implant 80 via the passage of filament through a pair of openings defined by a body of button implant 80. In the depicted example of button implant 80, these openings are in the form of eyelets. With reference to FIGS. 12A-13B, the button implant 80 defines a first eyelet 85A and a separate second eyelet 85B that extend from an open surface 86 to a contacting surface 88.

In example scenarios, the button implant 80 is configured to attach or couple to the instrument 10. In some examples, and as shown in at least FIGS. 12A-12B, the first end 81 of button implant 80, which is a proximal surface when attached to instrument 10, includes a base surface 83 and a slanted surface 84 adjacent to base surface 83. An edge 81A separates the respective surfaces. In the depicted embodiment, slanted surface 84 may be considered as a beveled or chamfered surface. In some examples, including that depicted in FIGS. 12A-12B, base surface 83 and slanted surface 84 are generally flat or planar. Edge 81A is one example of a biasing feature that promotes a predictable behavior of button implant 80 when button implant 80 interacts with instrument 10. A relationship between button implant 80 and instrument 10 is described in greater detail elsewhere in the present disclosure.

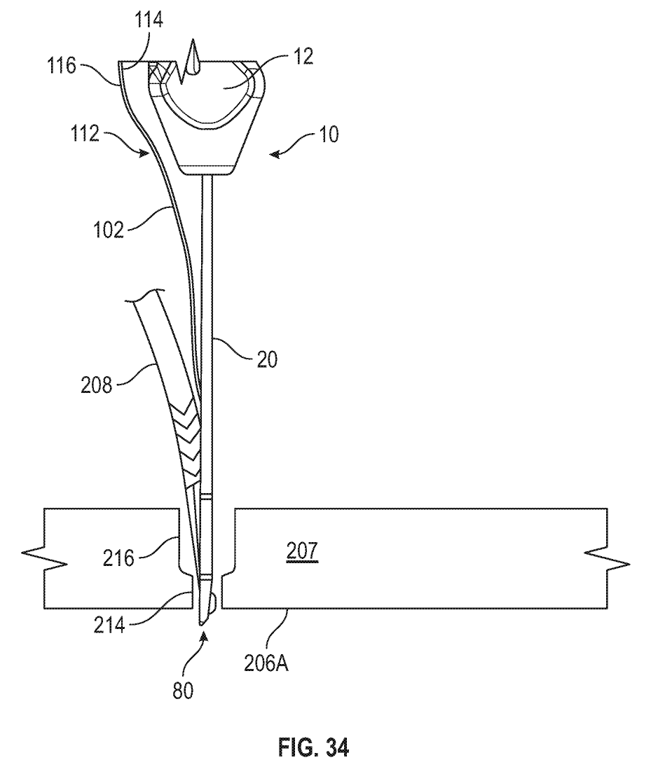

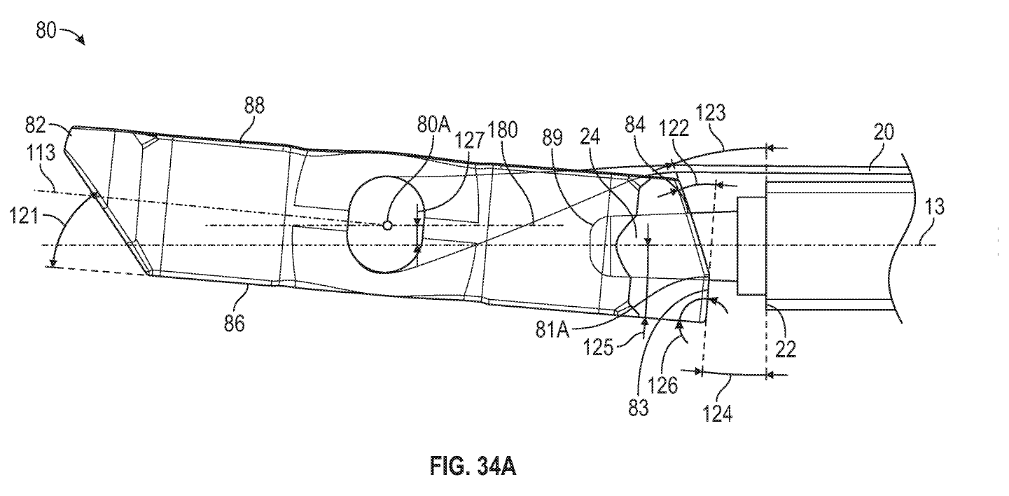

Turning to a relationship of the surfaces on button implant 80, as depicted in FIGS. 12A-12B, and with reference to FIG. 34A, an angle 126 between base surface 83 and open surface 86 is approximately 90 degrees, an angle 122 between base surface 83 and slanted surface 84 is approximately 23 degrees, and an angle 121 between open surface 86 and a surface on second end 82 is approximately 51 degrees. A shape of button implant 80 may be varied such that a shape of the surfaces at first end 81 may be different from that shown. For instance, in some examples, angle 126 may be any value from 70 degrees to 90 degrees. Further, in some examples, angle 122 may be any value from 10 degrees to 40 degrees. And, in some examples, angle 121 may be any value from 10 to 90 degrees. As to angle 121, the higher the angle within the aforementioned range, the greater the button strength. Conversely, the lower the angle within the aforementioned range, the greater the ability of the implant to penetrate tissue. Thus, in examples where angle 121 is in a range from 10-40 degrees, greater penetration functionality may be realized, and in examples where angle 121 is in a range from 40-90 degrees, the implant has greater structural integrity. In this manner, an angle 121 in a range from 35-45 degrees is advantageous in that it provides some of both the penetration and strength characteristics. In each of the aforementioned examples, first end may include edge 81A as described and shown. In other variations, first end may include a protrusion at a juncture of slanted surface and base surface. In still further variations, first end may have a single base surface with a protrusion thereon. In variations with a protrusion, the protrusion may be in between open surface 86 and contacting surface 88 and may be closer to one of those surfaces than the other or may be adjacent to one of those surfaces. The protrusion may have an angular or another shape and may be surrounded by one or more peripheral surfaces that are recessed relative to the protrusion. In each of the aforementioned examples and variations, edge 81A or protrusion may function as a pivot axis for button implant 80 during use, as described elsewhere in the present disclosure.

Further, as shown in the example depicted in FIGS. 12A-12B, the button implant 80 defines an opening 89 extending into an interior of button implant 80 from slanted surface 84. Opening 89 is large enough to receive an end portion, i.e., tip 24B of inner shaft 24 and, as depicted, may have a central longitudinal axis that is at a non-zero angle, i.e., non-parallel relative to a central longitudinal axis 113 of an elongate dimension of button implant 80. Another characteristic of opening 89 is that the central longitudinal axis of the opening 89 may be offset by an offset distance 127 from an axis 180 passing through a center 80A, e.g., a volumetric center of button implant 80, where axis 180 is parallel to the central longitudinal axis of the opening 89, as shown in FIG. 34A, for example. When button implant 80 is attached to instrument 10, such offset could also be said to be between center 80A and central longitudinal axis 13 of instrument 10. And, in some variations where center 80A is offset from central longitudinal axis 13 when button implant 80 is attached to instrument, opening 89 at first end 81, i.e., the proximal surface, is closer to one of open surface 86 and bone contacting surface 88 of button implant 80. Further, with continued reference to an attached configuration, at least part of the surfaces at first end 81 may be angulated relative to distal end surface 22 of outer shaft 20. In some variations, button implant 80 may define an opening 89 with a central longitudinal axis angled relative to a central longitudinal axis 113 of a length of button implant 80 (measured between first and second ends 81, 82). In some examples of such variations, a location of a center of opening 89 at slanted surface 84 (or another surface at first end 81) may be spaced apart from central longitudinal axis 113. In other examples, the location of the center of opening 89 at slanted surface 84 may be at central longitudinal axis 113. In other variations, the central longitudinal axis of opening 89 may be parallel to a central longitudinal axis 113 of button implant 80. When the central longitudinal axis of opening 89 is parallel to central longitudinal axis 113, the respective axes (i.e., of opening 89 and central longitudinal axis 113 of button implant 80) may be coincident with or offset from each other. In some variations of these examples, first end 81 of button implant may be a single surface. Such single surface may be flat from open surface 86 to bone contacting surface 88. Further, in some variations, a surface at first end 81 may be perpendicular to central longitudinal axis 113.

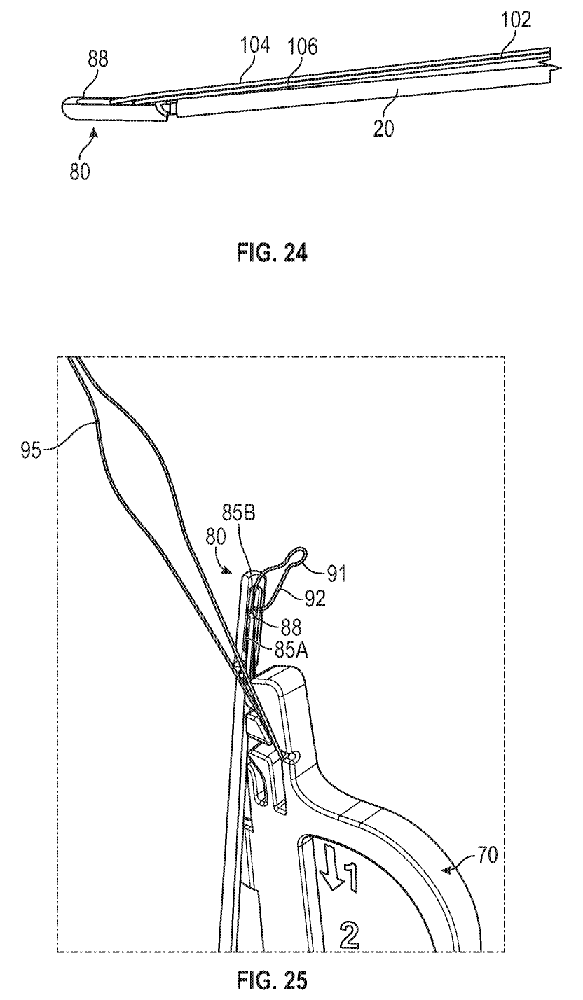

When button implant 80 is attached to instrument 10, base surface 83 and slanted surface 84 may be considered trailing surfaces, and overall, may be considered as a proximal surface. In this attached position, an angle 124 between base surface 83 and distal end surface 22 of outer shaft 20 may be approximately 5 degrees, and an angle 123 between slanted surface 84 and distal end surface 22 may be approximately 18 degrees, as shown in FIG. 34A. In variations, these angles may vary from the described example as a function of the specific surface characteristics of the surfaces on first end 81 of button implant 80. For instance, in one variation, central longitudinal axis 113 may be parallel to a central longitudinal axis of opening 89. The structure of button implant 80 includes several separate features that, when used in conjunction with a delivery instrument such as instrument 10, facilitate a release with flip such that the flip of button implant 80 occurs in a predictable manner. One such feature in button implant 80 as depicted is slanted surface 84 in combination with edge 81A, which may function as a pivot axis to induce button implant 80 to flip in a predetermined direction. Similarly, a button implant 80 structured and attached to instrument 10 in a manner such that angle 123 is greater than angle 124 may also be described as providing this functional advantage. Another feature is the offset distance 127 between the central longitudinal axis of opening 89, which dictates a position of button implant 80 relative to instrument 10 when button implant 80 is received on instrument 10, and axis 180 through center 80A of button implant 80 parallel to the central longitudinal axis of opening 89. This sets a relationship between button implant 80 and instrument 10 that induces a flipping action in a predetermined manner. Specifically, when a retention filament 102 is tensioned (described in the methods of the present disclosure), its connection to button implant 80 is proximate center 80A and therefore offset from an axis of the instrument 10 on which button implant 80 is attached. Accordingly, because such tension is applied in an offset manner, button implant 80 is preloaded to cause button implant 80 to flip in a predetermined manner. Additionally, a position of button implant 80 relative to instrument 10 when button implant 80 is attached to instrument 10 ensures that retention filament 102, when passed around cross-bar 87 and loaded onto instrument 10 as shown in FIG. 24, is on one side, i.e., a single side of instrument 10. This feature, when included in combination with at least one of the above-described features, also induces flipping of button implant 80 in a predetermined manner. Further details describing the flipping action of button implant 80 when released from instrument 10 are provided in the description of the methods of the present disclosure.

It should be understood that while the examples above describe the button implant 80 having various characteristics to promote a flipping action in a predetermined manner when the button implant 80 is released from the instrument 10, it should be appreciated that in other examples the button implant 80 and/or the instrument 10 may have other suitable components and/or characteristics.

For example, and as shown in FIGS. 13A-13B, in some examples the first end 81 includes a first extension or arm, shown as first arm 83A, and a second extension or arm, shown as second arm 83B. The first arm 83A is shown to extend from a first side of the first end 81, and the second arm 83B is shown to extend from a second side of the first end 81 opposite the first side, forming an opening, space, or groove therebetween, shown a groove 84A. In some examples, the groove 84A includes a generally flat or planar surface. In some examples, the groove 84A includes an angled surface, for example angled relative to the open surface and/or the contacting surface 88, to promote a predicable behavior of the button implant 80 when the button implant 80 interacts with the instrument 10, as described herein.

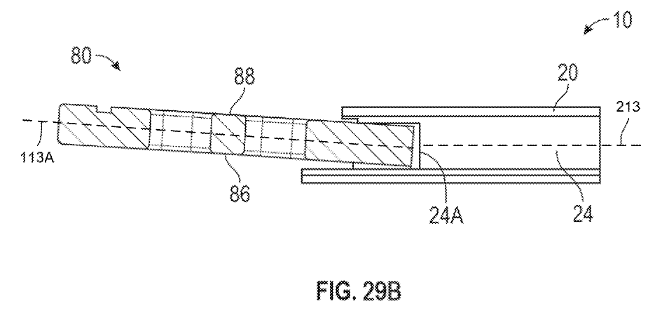

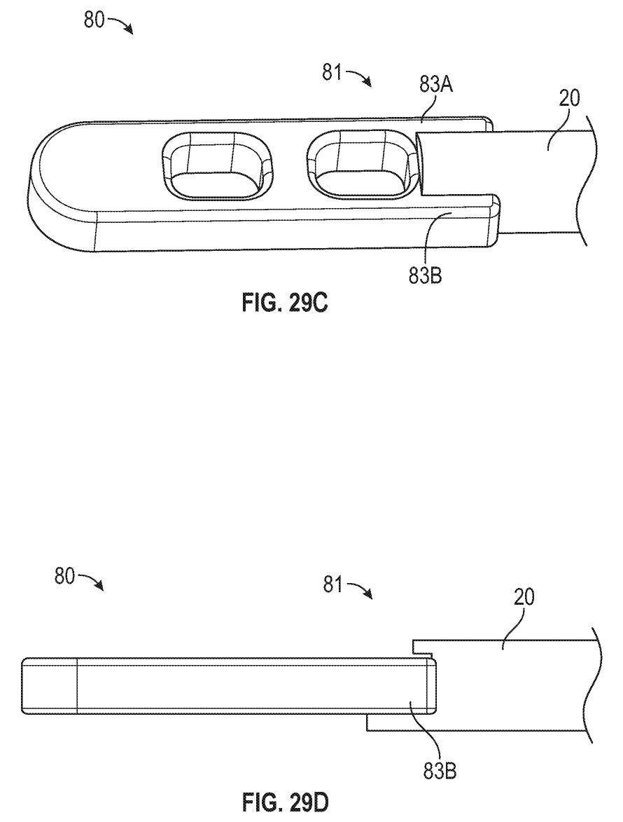

Turning to a relationship of the engagement between the button implant 80, as depicted in FIGS. 13A-13B, and with reference to FIGS. 29B-29D, the button implant 80 may be configured to engage an end of the instrument 10. In some embodiments, the button implant 80 includes one or more male connection or male interface components, and the instrument 10 includes one or more female connection or female interface components. For example, and as shown in at least FIGS. 29B-29D, in some examples an end portion of the instrument 10 includes one or more grooves or channels to engage the components of the first end 81. In some examples, an end or tip of the inner shaft 24 includes a first groove, shown as groove 24A. As shown in FIG. 29B, the groove 24A extends into the inner shaft 24, and is configured to receive the first end 81 of the button implant 80 such that the button implant 80 engages the inner shaft 24.

As shown in FIG. 29B, the groove 24A may be arranged such that when the button implant 80 is attached to the instrument 10, an angle exists between one or more axis and/or surfaces of the button implant 80 and a central axis 213 of the inner shaft 24. For example, and as shown in FIG. 29B, when the button implant 80 is attached to the instrument 10, an angle exists between an axis 113A, or a central longitudinal axis, of the button implant 80 and the central axis 213 of the inner shaft 24. In other examples, when the button implant 80 is attached to the instrument 10, an angle exists between surfaces of the button implant 80 (e.g., the open surface 86, contacting surface 88, etc.) and the central axis 213 of the inner shaft 24.

In some examples, to provide such an attachment configuration the groove 24A may extend in a non-parallel relationship relative to a central axis of an elongate dimension of button implant 80 (e.g., the groove 24A extends at an angle relative to the central axis of the inner shaft 24, etc.). In other examples, the groove 24A is sized such that a height and/or width of the groove 24A is larger than a height and/or width of the button implant 80, thereby forming a loose-fit engagement and allowing the button implant 80 to be attached to the instrument (e.g., the inner shaft 24) at an angle. In an example embodiment, the angle is 5 degrees; however, in other embodiments the angle is another suitable angle. In some examples, the angle is configured to facilitate the button implant 80 flipping in a predetermined manner, for example about a central axis, about an axis along an edge of the button implant 80, onto and/or facing a surface of a bone, etc. All such variations and combinations are contemplated herein.

It should be understood that while the above examples describe button implant 80 with a first end 81 having various characteristics to promote a flipping action in a predetermined manner when button implant 80 is released from instrument 10, it should be appreciated that in alternative examples, a system that includes an implant and instrument may include an instrument 10 with an outer shaft 20 having a distal end surface 22 with characteristics to promote the flipping action. These features (not shown) on outer shaft 20 may be in addition to, or an alternative to those described for button implant 80 above. Thus, the features may be on the button implant 80 alone, on the outer shaft 20 alone, or on a combination of both. In some examples, distal end surface 22 of outer shaft 20 may include a sloped surface, i.e., non-perpendicular to central longitudinal axis 13 of instrument 10. In some examples, distal end surface 22 may include two surfaces angled with respect to each other and joined along an edge. Some such examples may be arranged so that one of the surfaces is a chamfered surface. Further examples may include three or more surfaces on distal end surface 22. Any one of the aforementioned surfaces may be planar. In some examples, distal end surface 22 may include one or more protrusions that are raised relative to a remainder of distal end surface 22. In one example, distal end surface 22 may include a recessed portion sized to receive a protrusion on first end 81 of button implant 80. In each of the foregoing examples, these surface characteristics may exist along a ring-shaped surface surrounding the cannulation of outer shaft 20 to allow for the relative movement of inner shaft 24 and outer shaft 20. In some specific examples, first end 81 of button implant 80 may have a surface that is perpendicular to a length direction of button implant 80, and distal end surface 22 of outer shaft 20 may include a surface feature such as an edge or protrusion that functions as a pivot axis or location to cause the button implant 80 to flip in a predetermined manner when released from instrument 10.

In other examples, a distal end of the outer shaft 20 (e.g., at the distal end surface 22, etc.) includes one or more grooves or channels configured to engage the components of the first end 81 of the button implant 80. In this sense, the instrument 10 (e.g., the outer shaft 20, etc.) may include one or more female connection components, and the button implant 80 may include one or more male connection components to engage the female connection components of the instrument. For example, a distal end of the outer shaft 20 may include one or more groove or channels, similar to the groove 24A, which is/are configured to engage components of the first end 81 of the button implant 80, as described herein. In some examples, with the button implant 80 engaged with the outer shaft 20, the inner shaft 24 (e.g., an end of the inner shaft 24) may be configured to engage the first end 81 of the button implant 80 to promote an angled alignment of the button implant 80 relative to the outer shaft 20, as described herein. In some examples, the inner shaft 24 is configured to engage the first end 81 to disengage the first end 81 from the outer shaft 20, for example to release the button implant 80 from the instrument. In other examples, an end of the outer shaft 20, an end of the inner shaft 24, and/or a combination thereof, include grooves or channels, similar to the groove 24A, which is/are configured to engage components of the first end 81 of the button implant 80 to promote an alignment of the button implant 80 relative to the instrument 10, as described herein. All such variations and combinations are contemplated herein.

With continued reference to button implant 80 attached to instrument 10, an angle 125 between central longitudinal axis 113 and central longitudinal axis 13 of instrument 10 may be approximately 5 degrees. The angle 125, based on an angle between central longitudinal axis of opening 89 relative to central longitudinal axis 113 of button implant, promotes an ability of button implant 80 to catch tissue prior to being released from instrument 10.

Button implant 80 is made of biocompatible materials. In some examples, button implant 80 may be made of a metallic material such as titanium or stainless steel. In other examples, button implant 80 may be made of polymeric materials such as polyether ether ketone (PEEK), polyethylene, or bioactive fiber reinforced absorbable materials.

In some examples, an implant may be an all-suture anchor. In other examples, an implant may be an all-suture filament. In these examples, the implant may be configured to include openings for the receipt of working filament 112.

In another aspect, the present disclosure relates to a system for repairing or reapproximating two pieces of tissue. In some examples, one or both of such pieces of tissue may be a soft tissue (i.e., soft tissue to soft tissue or soft tissue to bone) and in other examples, both may be a hard tissue (i.e., bone to bone). The two pieces of tissue may be in a joint of a patient. The system may use a variety of filament materials to complete a procedure, such as a suture material or a biologic material. A biologic material may be collagen material, synthetic material, or a combination of both. In one example, a system includes instrument 10, loading tab 70 and an implant, such as button implant 80. Loading tab 70 may include wires 91, 95 attached thereto. The components of such system may be interconnected as follows. Loading tab 70 is attached to instrument 10 through engagement of first, second and third gripping projections 73A-C onto outer shaft 20, as shown in FIG. 7A, and/or the other projections of the loading tab 70 described herein (e.g., as shown in FIG. 8A, etc.). Engagement and disengagement of loading tab 70 is characterized by a snap-on engagement. When engaged, loading tab 70 is slidable along outer shaft 20 of instrument 10. When loading tab 70 includes wires 91, 95 attached thereto, such wires may be positioned as desired through first and second eyelets 85A, 85B of button implant 80. Such positioning may be as shown in FIG. 7A, described in greater detail elsewhere in the present disclosure, for example. Button implant 80 may be engaged to instrument 10 via receipt of opening 89 on button implant 80 over inner shaft 24, and/or the other engagement of the instrument 10 with the button implant 80 described herein. When instrument 10, loading tab 70 and button implant 80 are assembled and set up for use, as shown in FIG. 7A, for example, extension 74 of loading tab 70 is aligned with button implant 80 and body 72 is aligned with outer shaft 20 of instrument 10. As described in greater detail in the methods of the present disclosure, button implant 80 may be further secured as part of the system through use of a retention filament 102 passed through first and second eyelets 85A, 85B and held between upper locking block 52 and lower locking block 41, as shown, for example, in FIG. 38. In the depicted embodiment, when button implant 80 is engaged with inner shaft 24, as shown in FIGS. 7A and 34A, for example, an elongate dimension of button implant 80 is at an angle 125 relative to central longitudinal axis 13 of outer shaft 20. However, button implant 80 may be at another angle relative to central longitudinal axis 13 or parallel to it, as described above. Features including an offset of button implant 80 relative to central longitudinal axis 13 of instrument 10 and/or a surface feature on one or both of first end 81 of button implant 80 and distal end surface 22 of outer shaft 20 of instrument 10 (e.g., slanted surface 84 extending on one side from a pivot edge 81A, a protrusion on distal end surface 22 of outer shaft 20, etc.), among other features, cause button implant 80 to be predisposed to flip in a predetermined direction.

In one example, a system includes instrument 10 and loading tab 70 without any implant. In such example, loading tab 70 may be engaged with instrument 10 in the same manner as described for the above system example. In yet another example, a system includes an instrument 10 and an implant, such as a button implant 80.

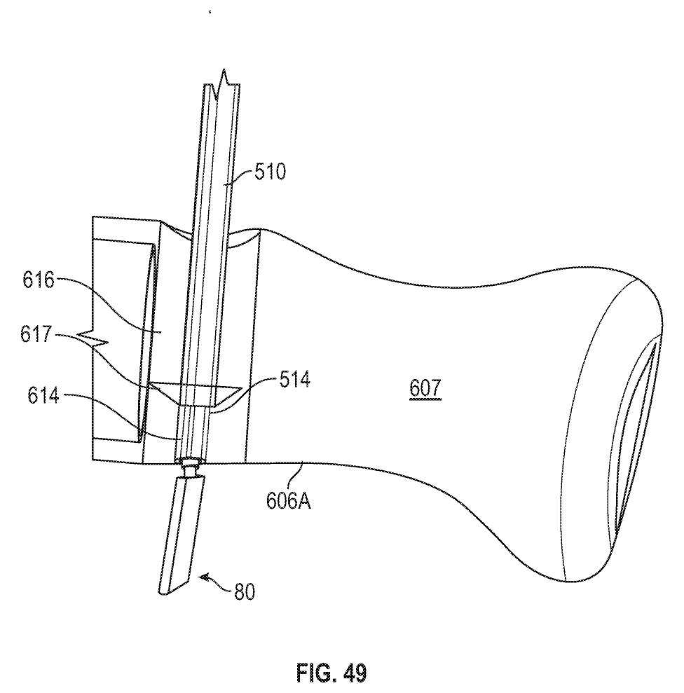

In any one of the above system examples that includes an instrument 10, a system may include a sleeve 510 as shown in FIGS. 48 and 49 that functions as a deployment depth control mechanism. Sleeve 510 includes a slot 512 along its length and has sufficient flexibility to be folded outward to snap into place onto an object, such as outer shaft 20. Through the compressive force of sleeve 510, once sleeve 510 is attached to outer shaft 20 it remains in place unless force is applied to translate it or otherwise remove it. Such sleeve may be used in the methods contemplated by the present disclosure.

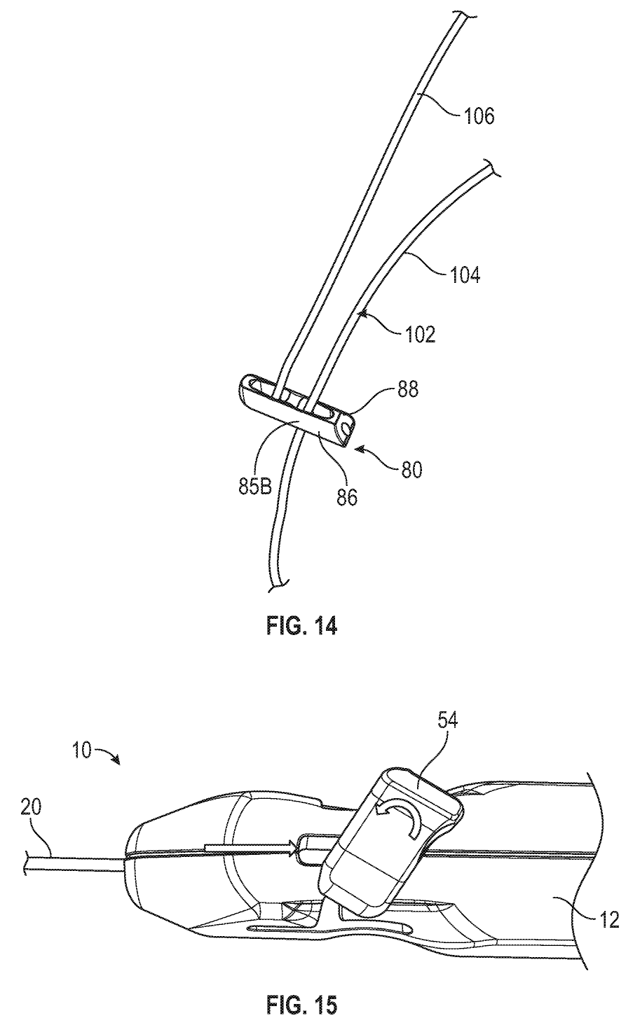

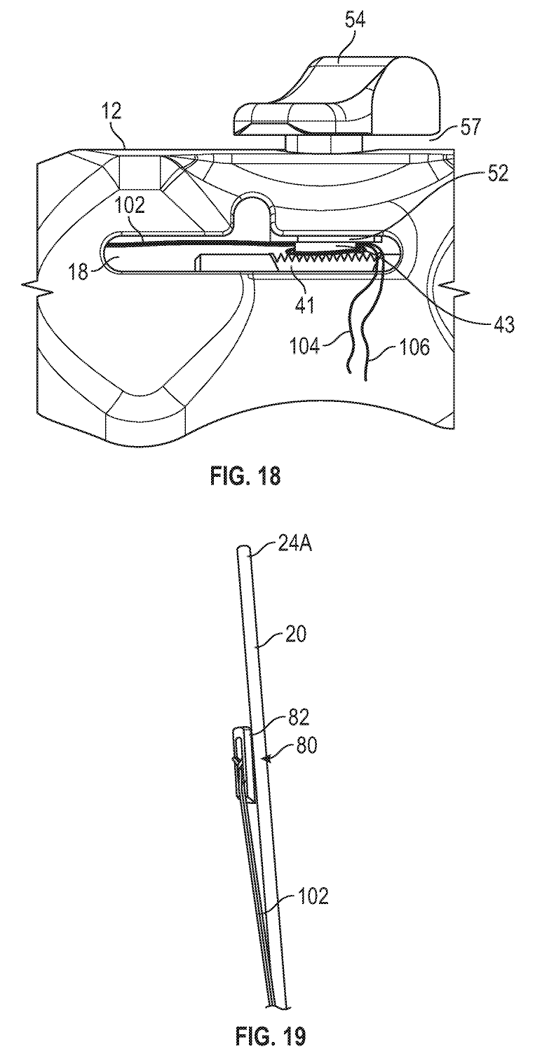

In any one of the above system examples, the system may include at least one of a retention filament 102 and a working filament 112. In systems that include a retention filament 102, such retention filament 102 may be loaded onto button implant 80 and handle 12 as shown in FIGS. 14-29A, described in greater detail elsewhere in the present disclosure. In other implementations, retention filament may be similarly loaded onto button implant 80 but instead of the positioning shown in the aforementioned figures, may be wrapped around gap 57 between handle tab 54 and a surface of handle 12, as shown in FIG. 18. In some variations of these examples, the system may include two or more retention filaments and/or two or more working filaments. In some examples, a retention filament 102 or a working filament 112 may be a size 6-0 suture, a size 0 suture, a size 5 suture, or any size in between. Suture tape, i.e., flat suture may also be used. In any number of examples, the retention filament and/or the working filament may have other sizes appropriate for the nature of the repair to be undertaken. A range of possible sizes of the filaments is limited by opening sizes in the implant used in conjunction with the filaments.

In another aspect, the present disclosure relates to a kit for repairing soft tissue in a joint. It is contemplated that the kit may include one or more of an instrument, a loading tab, an implant, a retention filament and a working filament, and that those kit components may be any of those contemplated by the present disclosure. In one example, a kit may include an instrument. In one example, a kit may include an instrument and a loading tab. In one example, a kit may include an instrument and an implant, such as a button implant. In yet another example, a kit may include an instrument, a loading tab and an implant. In any one of the aforementioned examples, a kit may include a sleeve to control deployment depth. In any one of the aforementioned examples, a kit may include a retention filament, a working filament, or both. In kits that include a loading tab, the loading tab may include one or more shuttles. A shuttle may be a wire or a suture, for example. In any one of the contemplated examples, a quantity of two or more of any of the aforementioned components may be included. Further, in any one of the contemplated examples, a kit may include any number of accessories used with an instrument of the kit to repair or reattach soft tissue. For example, a needle may be included in a kit. In one specific example, a kit may include an instrument 10, a button implant 80, a loading tab 70 with wires 91, 95, a retention filament 102, a drill pin, at least one suture for use as a whip loop suture (e.g., as shown in FIG. 30), and a needle. The contents of the kit may be provided in a single package or in a series of packages. In some instances, each component of a kit may be provided in a separate package. A kit may further be provided with an instruction manual with details on how to use the contents of the kit. In some examples, instructions may be printed on the packaging itself. In any one of the examples that includes packaging, the packaging itself may include indicia and/or surfaces shaped to organize and/or label different components of a kit.

In one aspect, the present disclosure relates to a method of preparing a system for use in repair or reattachment of soft tissue at a joint of a patient. In one example, a method of preparing a system for use in repair or reattachment of soft tissue is shown in FIGS. 14-29A. It should be appreciated that these method of preparation aspects may be performed at a manufacturing facility. Optionally, in some examples, the described method of preparing a system may be performed at a surgical site. The method may begin by preparation of a button implant 80 for loading onto an instrument 10. A retention filament 102 is retrieved, and may optionally be cut. In one example, a length of retention filament 102 for use with instrument 10 may be in a range from 400 mm to 500 mm. Preparation of button implant 80 includes passing an end from among first and second ends 104, 106 of retention filament 102 through respective first and second eyelets 85A, 85B of button implant 80 so that retention filament 102 loops over cross-bar 87 and such that the first and second ends 104 and 106 are on the same side as the contacting surface 88, as shown in FIG. 14.

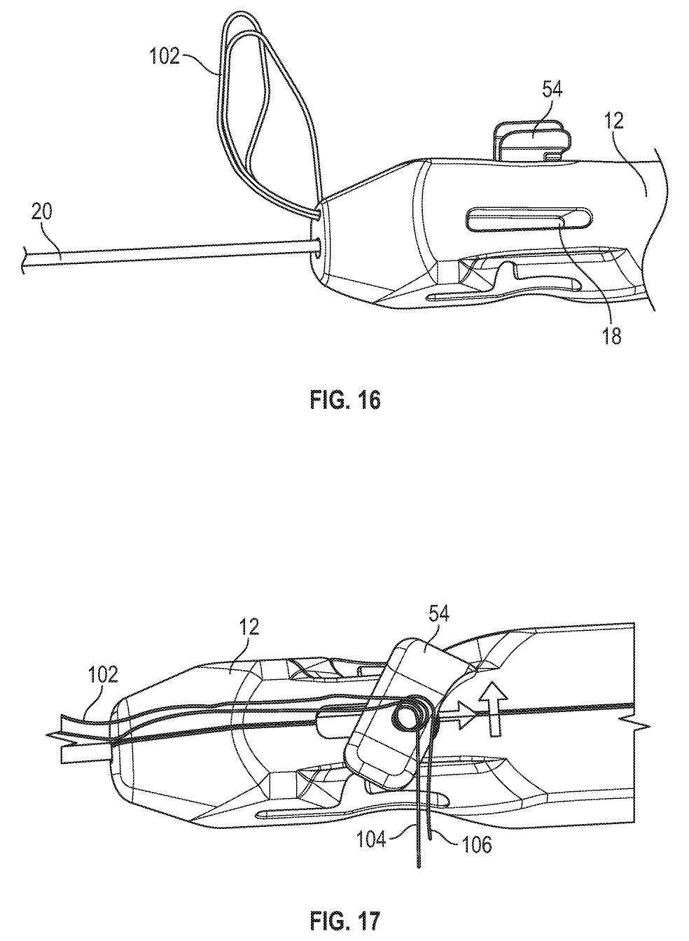

At any time before or after passing retention filament 102 through first and second eyelets 85A, 85B of button implant 80, handle tab 54 is retracted and rotated into an unlocked configuration, as shown in FIG. 15. In the depicted example, rotation is counterclockwise so that a long dimension of handle tab is transverse to an elongate dimension of handle 12 to arrive at the unlocked configuration. Each of first and second ends 104, 106 of retention filament 102 is then passed into a channel 67 at a distal end of handle 12 and, via channel 67, into upper cavity 69, as generally shown in FIG. 16. The channel 67 and upper cavity 69, without retention filament 102, are shown in FIG. 2. First and second ends 104, 106 are then pulled out of window 18 and are wrapped around inner block 43. When wrapped, retention filament 102 should be held and positioned adjacent to inner block 43 and in a space between opposing surfaces of upper locking block 52 and lower locking block 41. In this arrangement, retention filament 102 extends on a single side of button implant 80 and outer shaft 20 between button implant 80 and the actuation mechanism of instrument 10. In some examples, each of first and second ends 104, 106 of retention filament 102 are wrapped one and one quarter turns, or 450 degrees around inner block 43, as shown in FIGS. 17-18, and pulled out from window 18. It should be appreciated that, with respect to FIG. 17 in particular, a portion of retention filament 102 through handle 12 is shown as visible from above the handle 12 for illustration purposes only, and that an expected path of travel of retention filament 102 is depicted in FIG. 18. In variations, the retention filament 102 may be wrapped around inner block 43 more than one and one quarter turns. For example, retention filament may be wrapped two and one quarter turns, or 810 degrees, around inner block 43. A workable range of possible turns of retention filament 102 around inner block 43 may be limited at a low end by what is necessary to obtain a secure grip and at a high end by what is necessary to be able to release the filament without excessive difficulty.

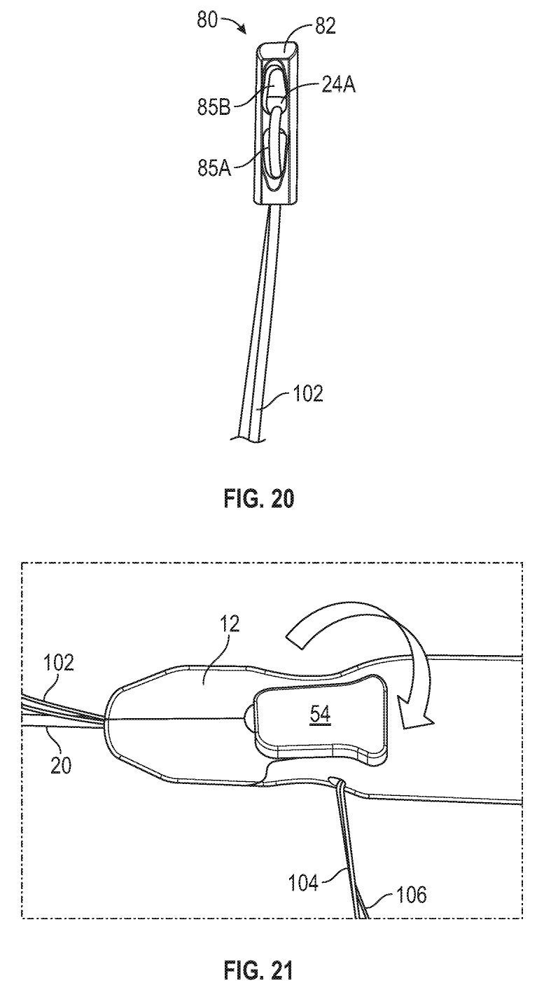

During the aforementioned process of wrapping retention filament 102 around inner block 43, first and second ends 104, 106 are pulled so that second end 82 of button implant 80 is approximately 25 mm short of tip 24B of inner shaft 24, as shown in FIG. 19. From this position, button implant 80 is pulled in a distal direction until second end 82 of button implant 80 is approximately 4 mm distal to tip 24B of inner shaft 24, as shown in FIG. 20. When button implant 80 is so positioned relative to inner shaft 24 (as shown in FIG. 20), handle tab 54 is rotated into a locked configuration, as shown in FIG. 21. In the depicted example, handle tab 54 is rotated clockwise into alignment with the elongate dimension of the handle to arrive at the locked position. Because retention filament 102 is wrapped closely around inner block 43, the effect of manipulating handle tab 54 to arrive in the locked configuration is to engage retention filament 102 via compression of upper locking block 52 against lower locking block 41, effectively pinching retention filament 102. If retention filament 102 is not secured between such lower and upper locking blocks 41, 52, the process should be repeated.

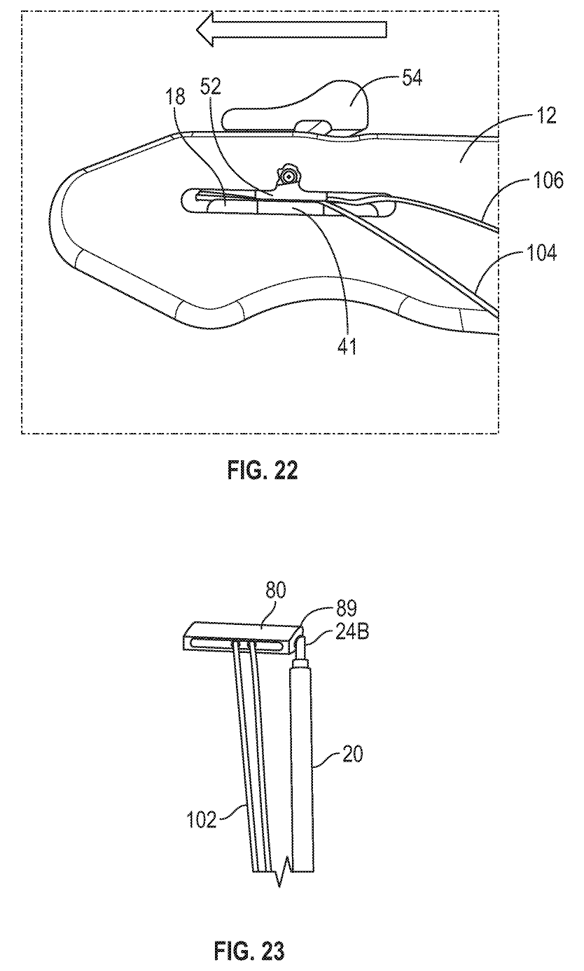

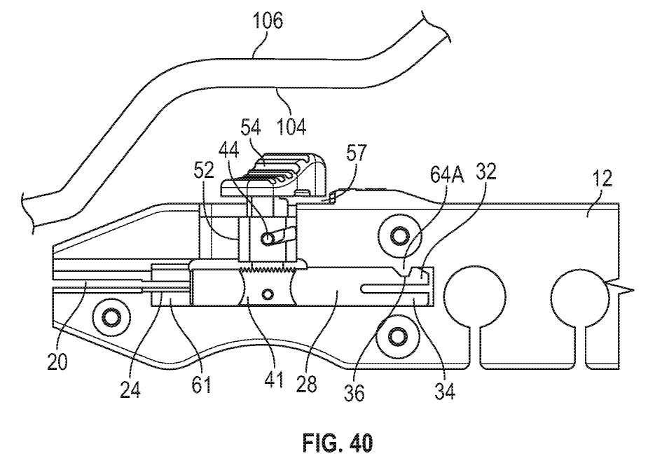

With instrument 10 in a locked configuration such that lower and upper locking blocks 41, 52 are engaged with first and second ends 104, 106 of retention filament 102, handle tab 54 is translated distally as shown in FIG. 22. This allows button implant 80 to be placed over tip 24B of inner shaft 24. Specifically, and as shown in FIGS. 23-24, button implant 23 is pulled over tip 24B of inner shaft 24 and opening 89 of button implant 80 receives tip 24B. At this juncture, button implant 80 should be oriented so that first and second ends 104, 106 of retention filament 102 extend from contacting surface 88 of button implant 80. As shown in FIGS. 7A and 24, it should be appreciated that when opening 89 receives tip 24B, an elongate dimension of button implant 80 is at a slight angle relative to central longitudinal axis 13 of outer shaft 20. Further, retention filament 102 should be evaluated to ensure it is taut. For example, when using #0 (zero) size retention filament, retention filament 102 should maintain a tensile force in a range of 5-50 N. Such force ensures that button implant 80, when deployed, flips as intended. If retention filament 102 is too loose or too tight, a tension in retention filament 102 may be adjusted as follows. Button implant 80 is disengaged from tip 24B, then handle tab 54 is rotated into an unlocked configuration. The aspects shown in FIGS. 19-24 may then be repeated. When lining up button implant 80 with inner shaft 24, button implant 80 should be excessively proximal in its position relative to inner shaft 24, and from there, button implant 80 may be pulled distally to bring button implant 80 over tip 24B. Such adjustment may be repeated, if necessary, until retention filament 102 is taut. In some examples, reloading of retention filament 102 may be accomplished without running first and second ends 104, 106 into the body of handle 12. This may be a preferred approach when it is not possible to feed retention filament 102 back into channel 67, for example. In such instances, first and second ends 104, 106 may be wrapped around gap 57 between handle tab 54 and the body of handle 12 (as shown in FIG. 40, for example) instead. A quantity of revolutions of the retention filament 102 around the post under handle tab 54 may be the same as contemplated for the depicted method, and the handle tab 54 may be rotated to hold retention filament 102 in place.

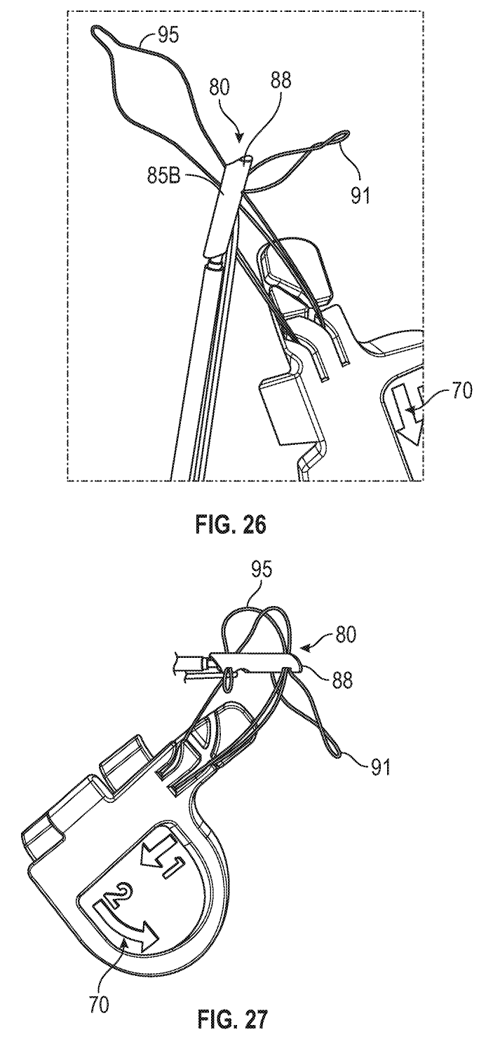

With retention filament 102 in position for use with instrument 10, loading tab 70 may be prepared for use with instrument 10 and button implant 80, as shown in FIGS. 25-28. First wire 91 is passed through first eyelet 85A from a side of contacting surface 88, and then around cross-bar 87 and through second eyelet 85B such that first loop 92 is on the side of contacting surface 88, as shown in FIG. 25. During the performance of this aspect, passage of wire through a loop of retention filament 102 is avoided. Second wire 95 is then passed through second eyelet 85B from the side of contacting surface 88, as shown in FIG. 26, then around cross-bar 87 and back through first eyelet 85A, as shown in FIG. 27, so that second loop 96 is on the same side as first loop 92 while also ensuring the second wire 95 is not passed through either the loop of retention filament 102 or the first loop 92 defined by first wire 91. Furthermore, second loop 96 of second wire 95 maintains a position of being on top of first wire 91, i.e., outside of first wire 91 throughout its path in the button implant 80 as it is subsequently positioned in fourth channel 78. A view of retention filament 102 and first and second wires 91, 95 from open surface 86 side of button implant 80 is shown in FIG. 29. For each of these aspects, care is taken to ensure that the respective first and second wires 91, 95 remain separate and never cross each other.

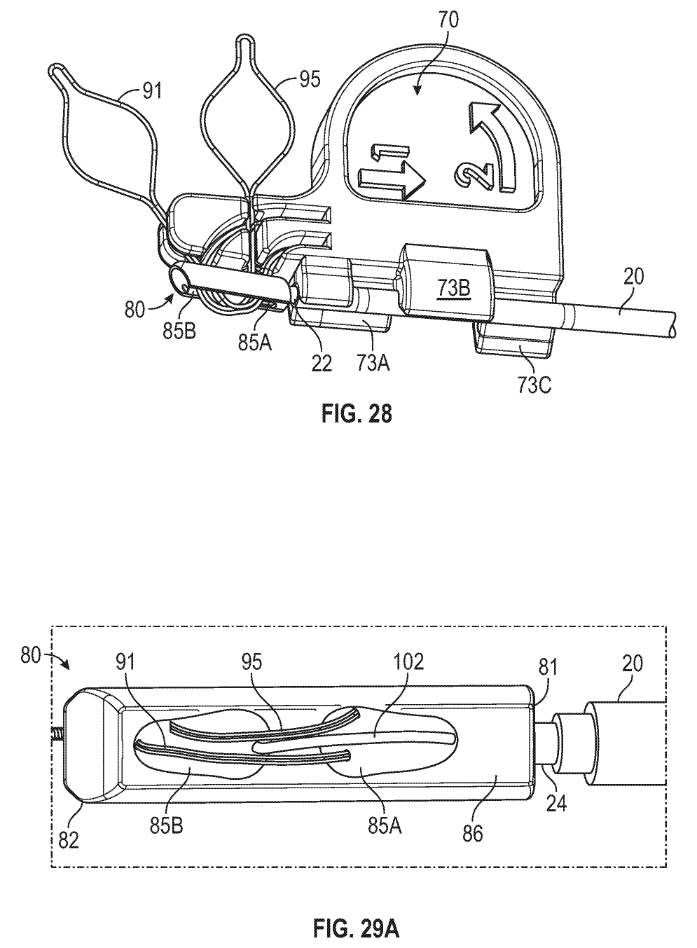

When each of first wire 91 and second wire 95 is in position through the eyelets in button implant 80, loading tab 70 is snapped onto outer shaft 20, as shown in FIG. 28. This ensures retention filament 102 is not pinned or smashed and thus will not be disrupted by the working filament 112 loading process. To finalize a position of first and second wires 91, 95 for use of instrument 10, loading tab 70 may optionally be slid distally along outer shaft 20 and first narrowed end 93 and second narrowed end 97 may be pulled so that respective first and second wires 91, 95 are taut and opened to their desired fullness to ease the process of loading the working filament 112. Additionally, each of first wire 91 and second wire 95 may optionally be positioned into respective channels on extension 74 of loading tab 70. Specifically, first wire 91 may be positioned in first channel 75 and third channel 77, while second wire 95 may be positioned in second channel 76 and fourth channel 78.

In some examples, the method of preparing a system for use in soft tissue repair or reattachment may be performed using the loading tab 70 of FIGS. 8A-8B and/or the button implant of FIGS. 13A-13B. Further, in some examples the first and/or second wires 91, 95 may be otherwise arranged or configured. All such combinations and variations are contemplated herein.

In some examples, the method of preparing a system for use in soft tissue repair or reattachment may be performed using loading tab 70` or loading tab 70``. Further, in some examples of examples with a loading tab, a pair of shuttles 491 may be used in place of first and second wires 91, 95. In some examples, the method may be performed without loading tab 70. In some examples, instrument 310 may be used in place of instrument 10.

Alternative arrangements of loading tab 70, 70`, 70`` are also envisioned. In one example, first and second wires 91, 95 on loading tab 70 are passed through first and second eyelets 85A, 85B in an opposite manner from that shown in FIGS. 7A, 28, and 29A, and described above. Specifically, from wire opening 79A on loading tab 70, first wire 91 extends through second eyelet 85B, then back through first eyelet 85A, exiting button implant 80 from a side of contacting surface 88. And, from wire opening 79B on loading tab 70, second wire 95 extends through first eyelet 85A, then back through second eyelet 85B, and exits button implant from the side of contacting surface 88.

In one aspect, the present disclosure relates to a method of using a system to repair or reattach soft tissue at a joint of a patient. The contemplated methods may be performed in many areas of the body, such as in the elbow, in the knee, in the shoulder, in the hip, and in the ankle among other joints. In one example, a method of using a system to repair or reattach soft tissue is shown in FIGS. 30-42. For the sake of clarity, the described method relates to repair of a biceps tendon 208, shown along with humerus 202, ulna 204 and radius 206 in FIG. 30. However, it should be appreciated that the method is not so limited and that reference to such anatomy is for the purposes of illustration.

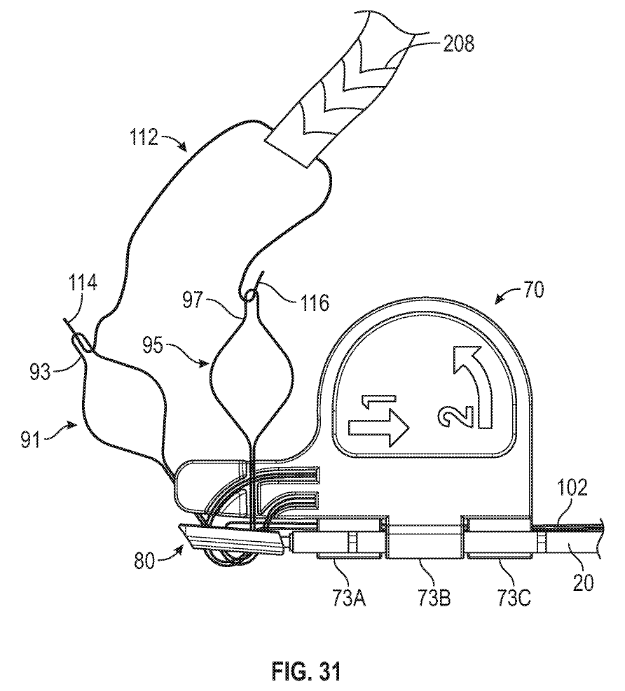

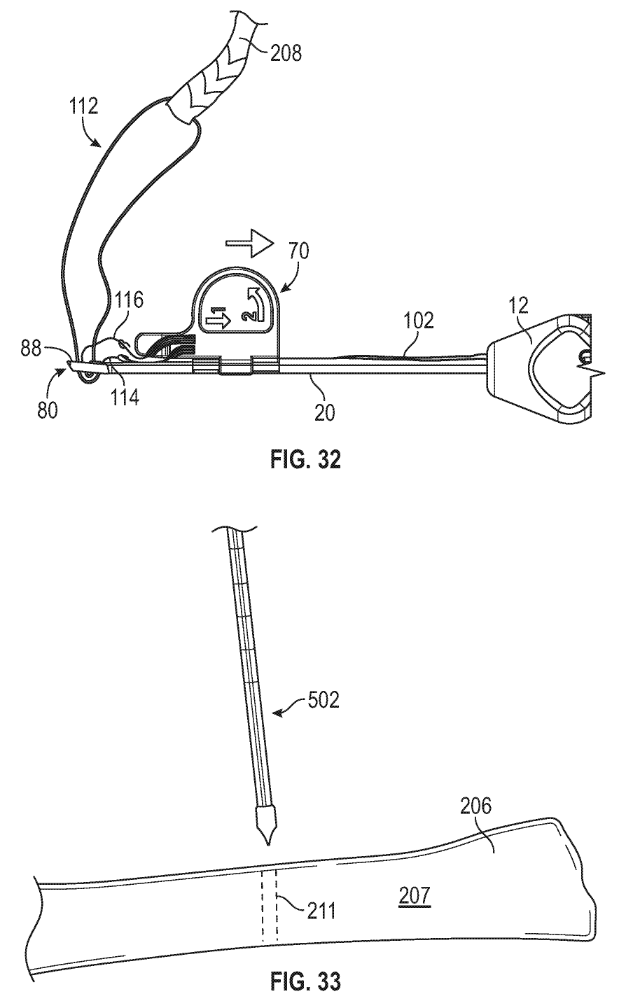

As shown in FIG. 30, the method begins with engagement of a working filament 112 to an end of a biceps tendon 208. A whip suture may be used to facilitate the engagement. Further, known techniques may be used to secure working filament 112 to biceps tendon 208. For example, the QuadCinch™ soft tissue preparation technique by Stryker® may be used. After engagement, first and second end portions 114, 116 of working filament 112 remain accessible for use. As shown in FIG. 31, first end portion 114 is then engaged with first narrowed end 93 of first wire 91 and second end portion 116 is engaged with second narrowed end 97 of second wire 95. Engagement may be accomplished simply by folding over ends of respective first and second end portions 114, 116 over first and second narrowed ends 93, 97. This is rendered easier because each of first and second wire 91, 95 is unobstructed, separated and clearly identifiable for engagement. As the method proceeds, and as shown in FIG. 32, loading tab 70 is slid proximally along outer shaft 20. This causes first and second wires 91, 95 to be drawn through the first and second eyelets 85A, 85B of button implant 80, in turn pulling respective first end portion 114 and second end portion 116 of working filament 112 along the same path. Specifically, first end portion 114 enters and passes through second eyelet 85B, passes over cross-bar 87, then enters and passes through first eyelet 85A. Second end portion 116 enters and passes through first eyelet 85A, passes over cross-bar 87, then enters and passes through second eyelet 85B. After first and second end portions 114, 116 are each pulled through the pair of eyelets, i.e., first and second eyelets 85A, 85B, an entirety of first and second wires 91, 95 is removed from button implant 80, leaving only working filament 112 passing through first and second eyelets 85A, 85B of button implant 80. In variations of the method, loading tab 70 is pulled diagonally away from button implant 80 or closer to a direction that is orthogonal relative to central longitudinal axis 13 of outer shaft 20 to draw first and second end portions 114, 116 through first and second eyelets 85A, 85B. This may also bring biceps tendon 208 towards a distal end of instrument 10. At this juncture, loading tab 70 may be removed from outer shaft 20. One advantage realized through the use of loading tab 70 with wires 91, 95 is that first and second end portions 114, 116 of working filament 112 are passed through the eyelets of button implant 80 in a predetermined manner without any passage through an unintended eyelet, filament loop, wire or other unintended and undesired location. This ensures proper filament passage in a first instance and thus avoids the need for passing the working filament 112 through the button implant 80 two or more times.

Next, a bone region that will receive the implant is prepared. As shown in FIG. 33, a drill 502, e.g., a drill pin, and, as applicable, other instruments (not shown) are used to prepare an opening to aid in the completion of the repair. In the depicted example, a through hole is prepared in the radial tuberosity 207 for the distal biceps tendon repair. Such hole may initially be a single diameter opening 211, i.e., a pilot hole, but may be further refined to create a space for implant and graft deployment. Additional hole preparation may be through the use of a cannulated reamer or other similar instruments. In the depicted example, the hole is refined to have a stepped shape, with a distal portion 214 having a smaller diameter than a proximal portion 216. In some examples, distal portion 214 may have a diameter of approximately 2-4 mm while proximal portion 216 may have a diameter of 6-8 mm. A determination of a size, e.g., diameter of proximal portion 216 may be made based on a preceding evaluation of a size of biceps tendon 208 using the sizing feature on instrument 10, described elsewhere in the present application. While first and second end portions 114, 116 of working filament 112 are held, instrument 10 may then be advanced to the hole, as shown in FIG. 34. Because this example involves positioning of button implant 80 on an outside surface of radius 206, instrument 10 may be inserted so that button implant 80 protrudes from the hole on a side of radius 206 opposite that through which instrument 10 is advanced into the hole.

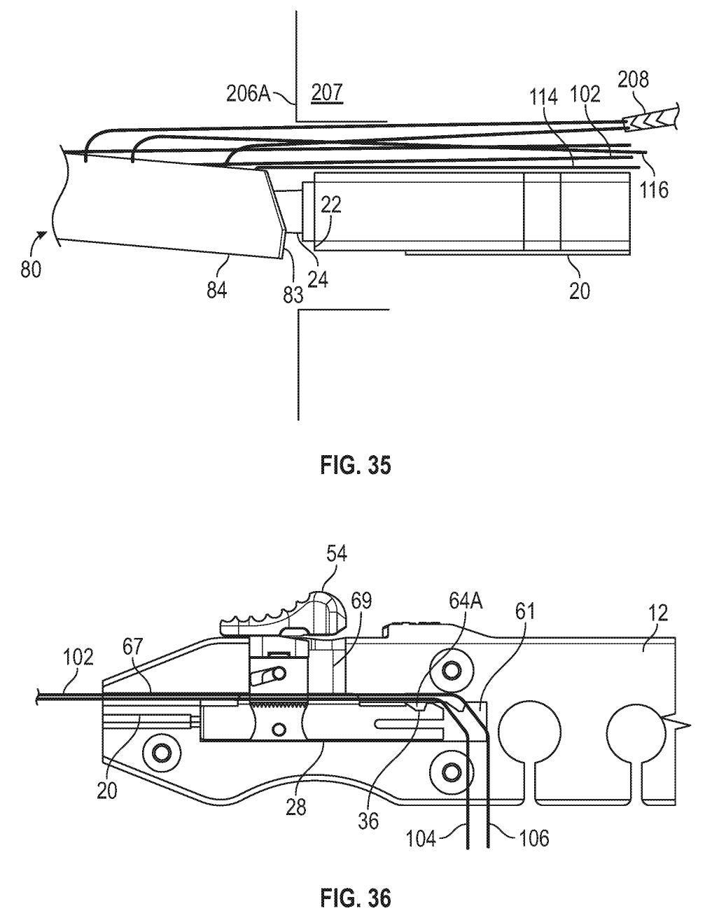

Once instrument 10 is in position in the hole at the radial tuberosity 207, the instrument 10 may be translated in a proximal direction (from a position approximating that shown in FIG. 35), thereby causing base surface 83 to come into contact with the desired bony surface 206A to gauge the proper location for activating the deployment of the button implant 80 such that it is not too far into tissues beyond the pilot hole and is far enough to allow proper button flipping/deployment. Then, proximal translation of handle tab 54 causes button implant 80 to flip onto bony surface 206A of radius 206. Specifically, and as shown in the sequence of FIGS. 35-40 and FIGS. 39A-39C, as handle tab 54 is translated proximally, first end 81 of button implant translates toward and then presses against distal end surface 22 of outer shaft 20, while tip 24B of inner shaft 24 begins to withdraw from within opening 89 of button implant 80. With further proximal translation of handle tab 54, a force of first end 81 of button implant 80 against distal end surface 22 increases until it is sufficient to cause button implant 80 to flip via a pivoting action along edge 81A, where edge functions as a pivot axis. The instrument 10 and button implant 80 combination is configured to cause the button implant 80 to flip in a predetermined manner based on or more structural features of the button implant 80 and the associated methodology. One reason is the offset position of button implant 80 relative to outer shaft 20 of instrument 10, i.e., a center 80A of button implant 80 is offset from central longitudinal axis 13 of instrument 10, as shown in FIG. 34A. Another reason is because of the manner in which the surfaces at first end 81 of button implant 80 press against distal end surface 22. First end 81 has slanted surface 84 adjoining base surface 83 along edge 81A. Additionally, yet another reason is the arrangement of retention filament 102 on one side of button implant 80 when loaded on instrument 10. The effect of these features is to provide a predetermined flip direction during release so that a user can expect a particular side of the button implant 80 to face a bone surface, e.g., bony surface 206A, after the flipping action.

During the process of translating handle tab 54 proximally to ultimately release the attached button implant 80 from the instrument 10 in a flip action, first and second ends 104, 106 of retention filament 102 remain held in place between lower locking block 41 and upper locking block 52. Handle tab 54 may be translated further after the release of button implant 80 until groove 36 of terminal portion 28 snaps into positioning projection 64A so that inner shaft 24 arrives in a so-called “soft-lock” position. In some examples of the method, the button may release as the groove 36 snaps into positioning projection 64A. In the intended method of use of instrument 10, a user brings handle tab 54 into the soft-lock position before unlocking handle tab 54. Once inner shaft 24 is in the soft-lock position, it is prevented from sliding distally. However, if a distally directed force is applied manually onto handle tab 54, inner shaft 24 may be forced out of the soft-lock position. In some circumstances, this aspect of translating handle tab 54 distally out of the soft-lock position may be used as an aspect in reloading the retention filament 102. Of course, under expected operating conditions, handle tab 54 will not be unlocked prior to releasing the button implant 80, which should occur during proximal translation while groove 36 is still distal to the soft-lock position or at the time groove 36 snaps into the soft-lock position, i.e., into positioning projection 64A.

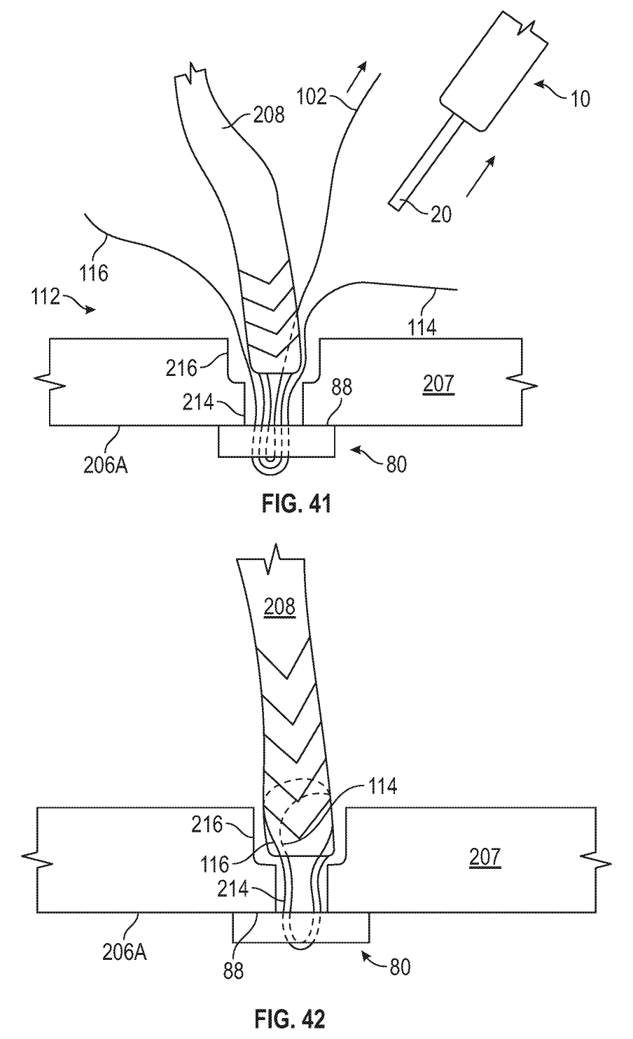

Retention filament 102 is then released from instrument 10. Specifically, handle tab 54 is rotated into the unlocked configuration, as shown in FIG. 40, then instrument 10 is withdrawn from the surgical site, as shown in FIG. 41. Once retention filament 102 is free of instrument 10, one end of first and second ends 104, 106 may be pulled to withdraw retention filament 102 entirely from button implant 80. First and second end portions 114, 116 of working filament 112 are then tensioned further to bring biceps tendon 208 into the hole in radial tuberosity 207. When biceps tendon 208 is in a desired repair position, first and second end portions 114, 116 are secured to biceps tendon 208. Securement may be through knots or other known techniques used to secure a filament to soft tissue.

In some examples, the method of using a system to repair or reattach soft tissue at a joint may be performed using loading tab 70` or loading tab 70``. In some examples, such method may be performed without a loading tab. In some examples, wires 91, 95 may be used without a loading tab. In some examples, wires 91, 95 may be replaced with a pair of shuttles 491. Stiff end portion 494 of respective shuttles 491 may be engaged to loading tab 70, 70`, 70``. In use, first and second end portions 114, 116 of working filament 112 may be received in respective hollow end portions 493 of shuttles 491 and then tensioned to form a finger trap. From there, the method may proceed as described above, i.e., respective shuttles 491 may be used to draw first and second end portions 114, 116 of working filament 112 through first and second eyelets 85A, 85B of button implant 80 in a predetermined manner. In any one of the above examples, the method of using a system to repair or reattach soft tissue may be performed with instrument 310 in place of instrument 10.

In still further examples, the method of using a system to repair or reattach soft tissue may include the use of sleeve 510 shown in FIGS. 48-49. In such examples, sleeve 510 is snapped onto outer shaft 20 so that a distal end surface 514 of sleeve 510 is at a set distance from distal end surface 22 of outer shaft 20. In some examples, an attachment position of sleeve 510 relative to outer shaft 20 may be predetermined based on interfacing features between sleeve 510 and outer shaft 20, e.g., a length of sleeve relative to outer shaft. In other examples, sleeve 510 may be configured to be attachable to outer shaft 20 in multiple locations along outer shaft 20 to provide different spacings between distal end surface 514 and an end of outer shaft 20. A position of attached sleeve 510 relative to an end of outer shaft 20 may then be used as a reference to establish a shape of the bone hole into which the instrument 10 will be passed. Specifically, a hole through bone 607 may be formed such that distal portion 614 of the hole has a smaller diameter than proximal portion 616 of the hole, as shown in FIG. 49. A user of the instrumentation may then insert the instrument until distal end surface 514 contacts end surface 617 of proximal portion 616 of the hole. At such time, the user will know that button implant 80 will be at an appropriate location relative to bone surface 606A for deployment. And, accordingly, a user may release and flip button implant 80 from instrument 10 when distal end surface 514 of sleeve 510 contacts end surface 617 within the bone with an expectation that button implant 80 will be in the proper position to be flipped into and properly seat on outer bone surface 606A. In other examples, these principles may be similarly applied to different deployment conditions.

In some examples, a method may include both preparing a system for use in soft tissue repair and performing the soft tissue repair. The aspects included in such a method may be any of those contemplated in the present disclosure.

In some examples, the method of soft tissue repair may be performed so that the implant is flipped internally within a bone hole or on another location on a bone other than that described for the depicted example. For example, instrument 10 may be advanced into a bone hole and while button implant 80 is inside the hole, the actuation mechanism of instrument 10 may be operated to flip button implant 80 in the predetermined manner so that button implant 80 is deployed inside the bone hole.

In some examples, the methods of soft tissue repair contemplated by the present disclosure may be performed by using retention filament 102 as a working filament, i.e., a filament to hold the implant to the tissue being repaired. In such a method, after the implant is deployed into an implanted position against tissue, first and second ends 104, 106 of retention filament 102 may be operatively engaged to the tissue being repaired, such as a biceps tendon. Engagement of retention filament 102 to the repair tissue may be through known techniques, and may include a knotless repair. In some variations, working filament 112 may be utilized in the method to bring the tissue under repair into position at a target site as described above, e.g., within a hole formed in a bone, but the retention filament 102 may be used to supplement the repair and/or complete the engagement between the tissue being repaired and the implant.

In some examples, the above methods may also be performed with the aid of robotics. For example, instrument 10 may be operatively connected with an end effector of a robotic system so that operation of instrument 10 may be controlled via computer. Such computer control may be real time or via execution of a predetermined surgical plan.

For any of the contemplated methods, it should be appreciated that the aspects described are not limited to being performed in the order as explicitly presented, and that one or more aspects of any one of the contemplated methods may be performed earlier or later in the method than the manner in which such aspect(s) is described herein. Similarly, the aspects of the contemplated methods should not be considered as a mandatory part of a method, and methods are contemplated that may omit one or more aspects of the methods as described. Further, the contemplated methods may also include one or more additional aspects beyond those that are described.

It is to be understood that the disclosure set forth herein includes any possible combinations of the particular features set forth above, whether specifically disclosed herein or not. For example, where a particular feature is disclosed in the context of a particular aspect, example, configuration, or arrangement, that feature may also be used, to the extent possible, in combination with and/or in the context of other particular aspects, examples, configurations, and arrangements of the contemplated apparatuses, systems, kits and methods. Non-limiting examples of the aforementioned features include implants, instruments, loading tabs, filaments and wires.

Although the disclosure herein has been described with reference to particular examples, it is to be understood that these examples are merely illustrative of the principles and applications of the present disclosure. It is therefore to be understood that numerous modifications may be made to the illustrative examples and that other arrangements may be devised without departing from the spirit and scope of the present disclosure as defined by the appended claims.

What is claimed:

1. A system for attaching soft tissue to a bone comprising:
an instrument including a handle, a movable component on the handle, and a shaft extending from the handle, the movable component being configured to control a portion of the shaft; and
a button implant releasably attached to a distal end of the shaft,
wherein manipulation of the movable component on the handle causes the button implant to be released from the instrument such that an end of the button implant remote from the instrument flips to a first or a second side of a central longitudinal axis of the shaft in a predetermined manner.

2. The system of claim 1, wherein the portion of the shaft is a first portion of the shaft, the first portion of the shaft being slidably disposed within a second portion of the shaft.

3. The system of claim 1, further comprising a retention filament removably engaged to the button implant and the movable component, the retention filament configured to pull the button implant releasably attached to the distal end of the shaft when the movable component is moved away from the button implant.

4. The system of claim 3, wherein the retention filament passes through a first eyelet of the button implant and a second eyelet of the button implant, and a first end portion of the retention filament and a second end portion of the retention filament are clamped by the movable component when the button implant is attached to the distal end of the shaft.

5. The system of claim 1, wherein a central longitudinal axis along an elongate dimension of the button implant is angled relative to the central longitudinal axis of the shaft.

6. The system of claim 5, wherein the elongate dimension of the button implant extends from a first end to a second end, the second end configured to attach to the shaft and having a surface at an oblique angle relative to a surface along the elongate dimension.

7. The system of claim 5, wherein the elongate dimension of the button implant extends from a first end to a second end, the second end including an opening therein sized to receive the portion of the shaft.

8. The system of claim 1, wherein the button implant has an elongate dimension extending from a first end surface to a second end surface, the second end surface including an opening therein sized to receive the shaft of the instrument, a central longitudinal axis of the opening being non-parallel to a central longitudinal axis along the elongate dimension of the button implant.

9. The system of claim 1, wherein the handle defines an opening sized to receive a soft tissue and measure the soft tissue, the opening being transverse to a length direction of the handle.

10. The system of claim 1, wherein the handle defines a plurality of openings, each opening of the plurality of openings passing through opposing outer surfaces of the handle such that each opening of the plurality of openings is oriented transverse to a length direction of the handle, the plurality of openings being sized to receive a soft tissue and measure the soft tissue.

11. The system of claim 10, wherein at least two openings of the plurality of openings have a different maximum cross-sectional dimension.
The system of claim 1, further comprising a sleeve configured to releasably attach onto the shaft of the handle such that the sleeve remains in position relative to the shaft when attached to the shaft.

13. The system of claim 12, wherein the sleeve includes a slot oriented along a length direction of the sleeve.

14. A method of attaching a soft tissue to a bone comprising:
passing first and second end portions of a working filament operatively engaged to the soft tissue through one or more eyelets of a button implant releasably attached to a distal end of a shaft of an instrument;
advancing the instrument into a hole in the bone;
manipulating an actuation mechanism on the instrument to cause a first elongate surface of the button implant to flip onto a bone surface of the bone as the button implant detaches from the shaft, wherein the first elongate surface is configured to flip with respect to the bone surface in a predetermined manner and a second elongate surface opposite the first elongate surface is configured to flip so that the second elongate surface faces away from the bone surface; and
securing the first and second end portions of the working filament to the soft tissue.

15. The method of claim 14, wherein the shaft is an inner shaft and manipulating the actuation mechanism includes pulling the actuation mechanism away from the button implant to cause the inner shaft to retract relative to an outer shaft of the instrument until the distal end of the inner shaft detaches from the button implant, and then pulling further such that a retention filament passing through the one or more eyelets of the button implant and attached to the instrument causes the button implant to flip.

16. The method of claim 15, further comprising, before the distal end of the inner shaft detaches from the button implant, drawing a trailing surface of the button implant toward an end of the outer shaft such that when the trailing surface contacts the outer shaft, an oblique angulation of the trailing surface relative to a distal end surface of the outer shaft induces the button implant to flip so that the first elongate surface faces the bone surface.

17. The method of claim 15, wherein manipulating the actuation mechanism is an initial manipulation and the method further comprises, subsequent to the initial manipulation, subsequently manipulating the actuation mechanism to cause the retention filament to be released from its attachment to the instrument.

18. The method of claim 17, wherein the initial manipulation comprises sliding the actuation mechanism away from the distal end of the shaft and the subsequent manipulation comprises rotating the actuation mechanism.

19. The method of claim 14, wherein prior to the passing step, the button implant is releasably attached to the shaft such that a central axis along an elongate dimension of the button implant is at an acute angle relative to a central axis of the shaft.

20. A system for attaching a first tissue to a second tissue comprising:
an instrument comprising:
a handle;
an outer shaft extending from the handle, the outer shaft including a distal end surface;
an inner shaft extending from the handle and disposed within the outer shaft; and
an actuation mechanism configured to control a position of one of the inner shaft and the outer shaft relative to the handle; and
an implant releasably attached to a distal end of the instrument, the implant including a proximal surface facing the distal end surface of the outer shaft,
wherein the system is configured such that when a filament is operatively engaged to the implant and the actuation mechanism, a relationship between the proximal surface of the implant and the distal end surface of the outer shaft causes the implant to flip in a predetermined manner upon actuation of the actuation mechanism.

21. The system of claim 20, wherein at least one of the proximal surface of the implant and the distal end surface of the outer shaft includes a chamfered surface.
